(19) United States
(12) Patent Application Publication
(10) Pub. No.: US 2024/0285615 A1
(43) Pub. Date: Aug. 29, 2024

(54) MEDICAMENTS COMPRISING GLYCOSIDASE INHIBITORS

(71) Applicant: Asceneuron SA, Lausanne (CH)

(72) Inventors: Dirk BEHER, Gland (CH); Bruno PERMANNE, Dingy-En-Vuache (FR); Rolf POKORNY, Flüh (CH); Anna QUATTROPANI, Rolle (CH)

(21) Appl. No.: 18/570,861

(22) PCT Filed: Jul. 5, 2021
(86) PCT No.: PCT/EP2021/068532

§ 371 Date: Dec. 15, 2023

(51) Int. Cl. A61K 31/496; A61P 1/00; A61P 9/10; A61P 25/08; A61P 25/28

(52) U.S. Cl. CPC A61K 31/496; A61P 1/00; A61P 9/10; A61P 25/08; A61P 25/28

(57) ABSTRACT

The present invention relates to pharmaceutical compositions and medicaments comprising the compound of formula (I)

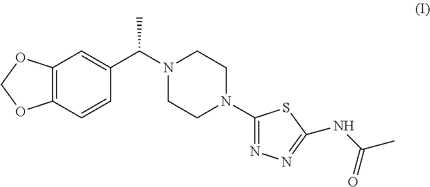 [SMILES: CC(=O)Nc1nnc(N2CCN([C@@H](C)c3ccc4c(c3)OCO4)CC2)s1]

or pharmaceutically usable solvates, salts or tautomers thereof, and dosage regimens for the administration thereof to human patients.

### FIELD OF THE INVENTION

The present invention relates to pharmaceutical compositions and medicaments comprising an O-GlcNAcase inhibitor and respective dosage regimens for the administration to human patients for the treatment of various disorders such as proteinopathies, including neurological disorders such as tauopathies, synucleinopathies and Alzheimer's disease.

### BACKGROUND OF THE INVENTION

A wide range of cellular proteins, nuclear, cytoplasmic and mitochondrial, are post-translationally modified by the addition of the monosaccharide 2-acetamido-2-deoxy-β-D-glucopyranoside (β-N-acetyl glucosamine) which is attached via an O-glycosidic linkage. This modification is generally referred to as O-linked N-acetylglucosamine or O-GlcNAc. The enzyme responsible for post-translationally conjugating β-N-acetylglucosamine (GlcNAc) to specific serine and threonine residues of numerous-cytoplasmic/nucleocytoplasmic proteins is O-GlcNAc transferase (OGT or OGTase). A second enzyme, known as O-GlcNAcase, removes this post-translational modification to liberate GlcNAc making the O-GlcNAc-modification a dynamic event occurring several times during the lifetime of a protein.

O-GlcNAc-modified proteins regulate a wide range of vital cellular functions including, for example, but not restricted to transcription, proteasomal degradation and cellular signaling. O-GlcNAc is also found on many structural proteins. For example, it has been found on a number of cytoskeletal proteins, including neurofilament proteins, synapsins, synapsin-specific clathrin assembly protein AP-3 and Ankyrin-G. O-GlcNAc modification has been found to be abundant in the brain. It has also been found on proteins clearly implicated in the etiology of several diseases including tauopathies, Alzheimer's disease (AD), synucleinopathies, Parkinson's disease, amyotrophic lateral sclerosis, and cancer.

For example, it is well established that AD and a number of related tauopathies including Down's Syndrome, progressive supranuclear palsy (PSP), Pick's disease, corticobasal degeneration (CBD), argyrophilic grain disease (AGD), globular glial tauopathy (GGT), frontotemporal dementia and parkinsonism linked to chromosome-17 (FTLD-17, Niemann-Pick Type C disease are characterized, in part, by the development of neurofibrillary tangles (NFTs). NFTs are also a histopathological hallmark of chronic traumatic encephalopathy that is a consequence of traumatic brain injury. These NFTs are aggregates of paired helical filaments (PHFs) and are composed of an abnormal form of the cytoskeletal protein “tau”. Normally, tau stabilizes a key cellular network of microtubules that is essential for distributing proteins and nutrients within neurons. In AD patients, however, tau becomes hyperphosphorylated, disrupting its normal function, forming PHFs and ultimately aggregating to form NFTs. Six isoforms of tau are found in the human brain. In AD patients, all six isoforms of tau are found in NFTs, and all are markedly hyperphosphorylated. Tau in healthy brain tissue bears only 2 or 3 phosphate groups, whereas those found in the brains of AD patients bear, on average, 8 phosphate groups. A clear parallel between NFT levels in the brains of AD patients and the severity of dementia strongly supports a key role for tau dysfunction in AD. The precise causes of this hyperphosphorylation of tau remain elusive. Accordingly, considerable effort has been dedicated toward: a) elucidating the molecular physiological basis of tau hyperphosphorylation; and b) identifying strategies that could limit tau hyperphosphorylation in the hope that these might halt, or even reverse, the progression of tauopathies, synucleinopathies and Alzheimer's disease. Several lines of evidence suggest that up-regulation of a number of kinases may be involved in hyperphosphorylation of tau, although very recently, an alternative basis for this hyperphosphorylation has been advanced.

In particular, it has recently emerged that phosphate levels of tau are regulated by the levels of O-GlcNAc on tau. The presence of O-GlcNAc on tau has stimulated studies that correlate O-GlcNAc levels with tau phosphorylation levels. The recent interest in this field stems from the observation that O-GlcNAc modification has been found to occur on many proteins at amino acid residues that are also known to be phosphorylated. Consistent with this observation, it has been found that increases in phosphorylation levels result in decreased O-GlcNAc levels and conversely, increased O-GlcNAc levels correlate with decreased phosphorylation levels. This reciprocal relationship between O-GlcNAc and phosphorylation has been termed the “Yin-Yang hypothesis” and has gained strong biochemical support by the recent discovery that the enzyme OGT forms a functional complex with phosphatases that act to remove phosphate groups from proteins. Like phosphorylation, O-GlcNAc is a dynamic modification that can be removed and reinstalled several times during the lifespan of a protein. Suggestively, the gene encoding O-GlcNAcase has been mapped to a chromosomal locus that is linked to AD. Hyperphosphorylated tau in human AD brains has markedly lower levels of O-GlcNAc than are found in healthy human brains. Very recently, it has been shown that O-GlcNAc levels of soluble tau protein from human brains affected with AD are markedly lower than those from healthy brain. Furthermore, PHF from diseased brain were suggested to lack completely any O-GlcNAc modification whatsoever. The molecular basis of this hypoglycosylation of tau is not known, although it may stem from increased activity of kinases and/or dysfunction of one of the enzymes involved in processing O-GlcNAc. Supporting this latter view, in both PC-12 neuronal cells and in brain tissue sections from mice, a nonselective N-acetylglucosaminidase inhibitor was used to increase tau O-GlcNAc levels, whereupon it was observed that phosphorylation levels decreased. Moreover, it has been described that the O-GlcNAc modification of tau directly inhibits its aggregation without perturbing the conformational properties of tau monomers. The implication of these collective results is that by maintaining healthy O-GlcNAc levels in AD patients, such as by inhibiting the action of O-GlcNAcase (OGA), one should be able to block hyperphosphorylation of tau and all of the associated effects of tau hyperphosphorylation, including the formation of NFTs and downstream effects. However, because the proper functioning of the lysosomal β-hexosaminidases is critical, any potential therapeutic intervention for the treatment of AD that blocks the action of O-GlcNAcase would have to avoid the concomitant inhibition of both lysosomal hexosaminidases A and B.

Consistent with the known properties of the hexosamine biosynthetic pathway, the enzymatic properties of O-GlcNAc transferase (OGT), and the reciprocal relationship between O-GlcNAc and phosphorylation, it has been shown that decreased glucose availability in brain leads to tau hyperphosphorylation. The gradual impairment of glucose transport and metabolism leads to decreased O-GlcNAc and hyperphosphorylation of tau (and other proteins). Accordingly, the inhibition of O-GlcNAcase should compensate for the age-related impairment of glucose metabolism within the brains of health individuals as well as patients suffering from AD or related neurodegenerative diseases.

These results suggest that a malfunction in the mechanisms regulating tau O-GlcNAc levels may be vitally important in the formation of NFTs and associated neurodegeneration. Good support for blocking tau hyperphosphorylation as a therapeutically useful intervention comes from studies showing that when transgenic mice harboring human tau are treated with kinase inhibitors, they do not develop typical motor defects and, in another case, show a decreased level of insoluble tau. These studies provide a clear link between lowering tau phosphorylation levels and alleviating AD-like behavioral symptoms in a murine model of this disease.

There is evidence indicating that the modification with O-GlcNAc may have a general function in preventing harmful protein aggregation. This has been directly demonstrated for the tau protein and also for the protein alpha-synuclein that is a toxic aggregating protein associated with synucleinopathies, including Parkinson's disease. Further aggregating proteins that are associated with amyotrophic lateral sclerosis [Tar DNA binding protein-43 (TDP-43) and superoxide-dismutase I (SOD-I)] and frontotemporal lobar degeneration (TDP-43) are known to carry the O-GlcNAc modification. These results indicate that increasing O-GlcNAcylation with OGA inhibitors could be in general beneficial in diseases associated with protein aggregations or, preferably, other protein misfolding and the resulting diseases or conditions, i.e. proteinopathies.

There is also a large body of evidence indicating that increased levels of O-GlcNAc protein modification provides protection against pathogenic effects of stress.

Humans have three genes encoding enzymes that cleave terminal β-N-acetyl-glucosamine residues from glycoconjugates. The first of these encodes the enzyme (protein)-3-O—(N-acetyl-D-glucosaminyl)-L-serine/threonine N-acetylglucosaminyl hydrolase (O-GlcNAcase). O-GlcNAcase is a member of family 84 of glycoside hydrolases. O-GlcNAcase acts to hydrolyze O-GlcNAc at serine and threonine residues of post-translationally modified proteins. Consistent with the presence of O-GlcNAc on many intracellular proteins, the enzyme O-GlcNAcase appears to have a role in the etiology of several diseases including type II diabetes, AD and cancer. Although 0-GlcNAcase was likely isolated earlier on, about 20 years elapsed before its biochemical role in acting to cleave O-GlcNAc from serine and threonine residues of proteins was understood. More recently O-GlcNAcase has been cloned, partially characterized, and suggested to have additional activity as a histone acetyltransferase.

### SUMMARY OF THE INVENTION

The present invention relates to pharmaceutical compositions and medicaments comprising the compound of formula (I)

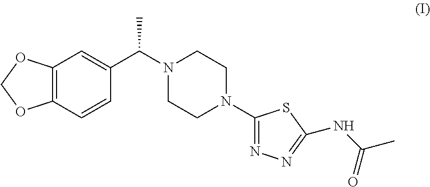 [SMILES: CC(=O)Nc1nnc(N2CCN([C@@H](C)c3ccc4c(c3)OCO4)CC2)s1]

and/or its tautomers in the respective free base form or in form of a respective pharmaceutically usable solvate or salt and dosage regimens for the administration thereof to human patients. Moreover, the present invention relates to a compound of formula (I) and/or its tautomers for use in a method of treating a human subject, the method comprising the repeated administration of one or more unit dosage forms comprising the compound of formula (I) and/or its tautomers in a suitable dose and at a suitable daily dosing frequency more specifically disclosed herein below. In a further embodiment, the invention relates to the use of a compound of formula (I) and/or its tautomers in the manufacture of a medicament for the treatment of a human subject, comprising the repeated administration of one or more unit dosage forms comprising the compound of formula (I) and/or its tautomers in a suitable dose and at a suitable daily dosing frequency more specifically disclosed herein below. Compound (I) and its use as glycosidase inhibitor is e.g. disclosed in WO 2016/030443. Objects of the invention comprise medicaments and pharmaceutical compositions comprising the compound of formula (I) and/or its tautomers and dosage regimens for the treatment of neurological disorders such as tauopathies, synucleinopathies and Alzheimer's disease.

### DESCRIPTION OF FIGURES

FIG. 1: Arithmetic Mean Plasma Concentration of Compound (I) Following a Single Oral Dose of Compound (I) at Seven Dose Levels (20 mg, 40 mg, 80 mg, 160 mg, 300 mg, 600 mg, and 1000 mg) in Healthy Male Subjects—Part 1a

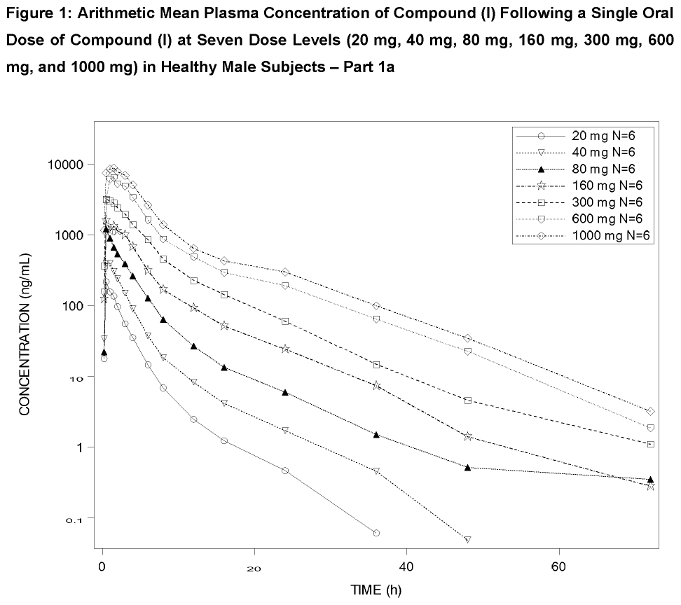

FIG. 2: Arithmetic Mean Plasma Concentration of Compound (I) Following BID Doses of Compound (I) at Dose Levels of 100 mg, 250 mg, and 500 mg in Male and Female Elderly Subjects for 12 Days at Day 12—Part 2

FIG. 3: Arithmetic Mean CSF Concentration of Compound (I) Following BID Doses of Compound (I) at Dose Levels of 100 mg, 250 mg, and 500 mg in Male and Female Elderly Subjects for 12 Days at Day 12—Part 2

FIG. 4: Integral PET Scan Images of Subject 1004: Baseline, 6.9 h after the Administration of 500 mg Compound (I) and 8.1 h after Administration of 300 mg Compound (I)

FIG. 5: First readout: PK-RO (Plasma Concentration versus Receptor Occupancy) Analysis. Curve Calculated According to

RO=100*Cp*/(Cp+EC50)

where RO=Receptor Occupancy, Cp=Plasma Concentration of Compound (I), EC50=is Cp Corresponding to 50% RO

FIG. 6: Dissolution Profile of Capsule and Tablet of Compound (I)

### DETAILS OF THE INVENTION

According to the invention, the medicaments and pharmaceutical compositions comprising the compound of formula (I), and dosage regimens for its administration to humans is particularly suited for the prophylactic or therapeutic treatment and/or monitoring of diseases that are caused, mediated and/or propagated by O-GlcNAcase activity. Diseases included in the present scope of the present invention are neurological and neurodegenerative diseases, diabetes, cancer, cardiovascular diseases and stroke, more preferably neurodegenerative diseases, most preferably one or more synucleinopathies and tauopathies, highly preferably Alzheimer's disease and dementia.

Further diseases to which the compounds of formula (I), or medicaments or pharmaceutical compositions comprising formula (I), can be applied in prophylaxis or therapy according to the invention include diseases or conditions selected from one or more proteinopathies. Such proteinopathies are preferably selected from the group comprising tauopathies, such as but not limited to Alzheimer's disease (AD), corticobasal degeneration (CBD), progressive supranuclear palsy (PSP), chronic traumatic encephalopathy or synucleinopathies (also called α-synucleinopathies), such as but not limited to Parkinson's disease, multiple system atrophy.

Another aspect of the invention relates to a method for treating neurodegenerative diseases, sleep disorders, such as insomnia, and neuropsychiatric conditions including depression and schizophrenia, diabetes, cancer, cardiovascular diseases and stroke, preferably a tauopathy, wherein a medicament or pharmaceutical compositions comprising the compound of formula (I) and/or physiologically acceptable salts thereof is administered according to the dosage regimens disclosed herein to a human in need of such treatment. The preferred way of administration is an oral administration. Medicaments and pharmaceutical compositions according to the invention, comprising the compound of formula (I) are especially preferred.

The neurodegenerative disease or condition is more preferably selected from the group of one or more tauopathies, synucleinopathies and Alzheimer's disease (AD), amyotrophic lateral sclerosis (ALS), amyotrophic lateral sclerosis with cognitive impairment (ALSci), argyrophilic grain disease, behavioral variant frontotemporal dementia (bvFTD), non-fluent and semantic variant primary progressive aphasia (nfv & svPPA), Bluit disease, corticobasal degeneration (CBD), Dementia pugilistica, Dementia with Lewy Bodies (DLB), diffuse neurofibrillary tangles with calcification, Down's syndrome, Familial British dementia, Familial Danish dementia, frontotemporal dementia with parkinsonism linked to chromosome 17 (FTDP-17), frontotemporal lobar degeneration (FTLD), ganglioglioma, gangliocytoma, Gerstmann-Straussler-Scheinker disease, globular glial tauopathy, Guadeloupean parkinsonism, Hallevorden-Spatz disease (neurodegeneration with brain iron accumulation type 1), lead encephalopathy, lipofuscinosis, meningioangiomatosis, multiple system atrophy (MSA), myotonic dystrophy, Niemann-Pick disease (type C), Pallido-ponto-nigral degeneration, Parkinson's disease, Parkinson's disease dementia (PDD), Parkinsonism-dementia complex of Guam, Pick's disease (PiD), postencephalitic parkinsonism (PEP), Prion diseases (including Creutzfeldt-Jakob Disease (GJD), variant Creutzfeldt-Jakob Disease (vCJD)), fatal Familial Insomnia, Kuru, progressive supercortical gliosis, progressive supranuclear palsy (PSP), pure autonomic failure, Richardson's syndrome also named PSP Richardson's syndrome, subacute sclerosing panencephalitis, Tangle-only dementia, tuberous sclerosis, Huntington's disease or mild cognitive impairment (MCI), Chronic traumatic encephalopathy, Primary progressive aphasia, Progressive nonfluent aphasia, Semantic dementia, Steele-Richardson-Olszewski syndrome, epilepsy, chronic and acute inflammation such as Crohn disease, neuroinflammation, subarachnoid hemorrhage (SAH), multiple sclerosis (MS), progressive forms of MS, such as primary or secondary progressive MS, Friedreich's Ataxia and Adenoleukodystrophy. Most preferred are one or more tauopathies, PSP, synucleinopathies, Parkinson's disease and Alzheimer's disease.

It has now been found that the compounds of formula (I) or pharmaceutically usable solvates, salts or tautomers thereof, can be advantageously administered to a human subject such as a patient in need thereof, following the dose and dosage regimens according to the present invention.

Throughout the specification, a dosage regimen according to the invention refers to or comprises the repeated administration of one or more unit dosage forms comprising the compound of formula (I) and/or its tautomers or pharmaceutically usable solvates or salts thereof to a human subject, in a suitable dose and at a suitable daily dosing frequency.

Preferably, a dosage regimen according to the invention refers to a method of treating a human subject, the method comprising the repeated administration of one or more unit dosage forms comprising the compound of formula (I) and/or its tautomers or pharmaceutically usable solvates or salts thereof in a suitable dose and at a suitable daily dosing frequency.

The invention also relates to the use of the compound of formula (I) and/or its tautomers and the dose regimens disclosed herein for the treatment of and a method of treating retinal degenerative diseases, preferably selected from glaucoma, age-related macular degeneration (AMD), retinitis pigmentosa and diabetic retinopathy.

The invention also relates to the use of the compound of formula (I) and/or its tautomers and the dose regimens disclosed herein for the treatment of and a method of treating inflammatory bowel diseases, preferably selected from colitis, such as ulcerative colitis, and Cohn's disease.

It is to be understood that the disclosure relating to the medical use of a compound per se shall apply analogously to the use of that compound when comprised in a pharmaceutical composition or medicament, or when that compound is used in a method of treatment or dose regimen as disclosed herein. The same applies in the converse, for instance it is understood that disclosures of the use of a compound as comprised in a pharmaceutical composition, in a medicament, or in a method of treatment involving administration of a compound, or as part of a dose regimen, shall apply analogously to medical uses of that compound per se.

As used herein the term “about” when referring to a particular value, e.g. an endpoint or endpoints of a range, encompasses and discloses, in addition to the specifically recited value itself, a certain variation around that specifically recited value. Such a variation may for example arise from normal measurement variability. The term “about” shall be understood as encompassing and disclosing, in addition to the exact referenced value itself, a range of variability above and below an indicated specific value, said variability being relative to the specific recited value itself, for example: The term “about” may encompass and disclose variability of ±5.0%. The term “about” may encompass and disclose variability of ±4.5%. The term “about” may encompass and disclose variability of ±4.0%. The term “about” may encompass and disclose variability of ±3.5%. The term “about” may encompass and disclose variability of ±3.0%. The term “about” may encompass and disclose variability of ±2.5%. The term “about” may encompass and disclose variability of ±2.0%. The term “about” may encompass and disclose variability of ±1.5%. The term “about” may encompass and disclose variability of ±1.0%. The term “about” may encompass and disclose variability of ±0.5%. Unless stated otherwise, where the term “about” is recited before the first endpoint of a numerical range, but not before the second endpoint of that range, this term, and the variability it implies in disclosure, refers to both the first endpoint of the range and the second endpoint of the range. For instance, a recited range of “about X to Y” should be read as “about X to about Y”. It is also understood that when the term “about” is applied to both the upper and lower endpoints of a range, different degrees of variability may apply for the upper and lower endpoints of the same range. All such possible different degrees of variability are also within the disclosure of the present application. For instance, in a range of “about X to about Y”, the application discloses i.a. a range in which the lower endpoint X varies within a tolerance of 1.5% of the stated value, while the upper endpoint varies within a tolerance of 2% of the stated value. Of course, this is merely exemplary, it being understood that all combinations of tolerances as set out above are included in the disclosure of the present application.

The term “human subject” is preferably taken to mean a patient having a disease or a human subject being at increased risk of acquiring a disease, wherein diseases are preferably selected from the diseases mentioned throughout this specification. More preferably, the term “human subject” is taken to mean a patient having a condition selected from neurological disorders or neurodegenerative diseases, diabetes, cancer, cardiovascular diseases and stroke, or a human subject being at increased risk of acquiring said disorders or diseases.

Preferably, the risk of acquiring a neurological disorder or neurodegenerative disease, such as PSP or Alzheimer is in some embodiments, a subject can be identified as being at increased risk of developing a neurological disorder or neurodegenerative disease, such as PSP, or identified as having a neurological disorder or neurodegenerative disease, such as e.g., at least in part, by detecting a genetic alteration in a gene encoding the microtubule-associated protein tau (MAPT) (e.g., any of the inversion polymorphisms in the MAPT gene, any of the haplotype-specific polymorphisms in the MAPT gene, the rare-coding MAPT variant (A152T), or mutations that enhance splicing of exon 10 in the MAPT gene described, e.g., in Hoglinger et al., Nature Genet. 43:699-705, 2011, and Hinz et al., Cold Spring Harb. Perspect Biol.). Non-limiting examples of genetic alterations in a gene encoding MAPT include mutations that result in the production of MAPT protein that include one or more point mutations of: S285R, L284R, P301L, and G303V. Additional specific genetic mutations in a gene encoding MAPT protein that can be used to identify a subject as having an increased risk of developing a neurological disorder or neurodegenerative disease, such as PSP, or can be used to identify a subject as having a neurological disorder or neurodegenerative disease, such as PSP are described in, e.g., Boxer et al., Lancet 16:552-563, 2017.

In some embodiments, a subject can be identified as being at increased risk of developing a neurological disorder or neurodegenerative disease, such as Alzheimer by genetic alterations in the presenilin 1 (PSEN1) or 2 (PSEN2) or β-amyloid precursor protein (APP) genes or by the presence of the E4 allele of the apolipoprotein E (APOE) gene or by having relatives bearing these alterations in their genes.

In some embodiments, a subject can be identified as having an increased risk of developing a neurological disorder or neurodegenerative disease, such as PSP or identified as having a neurological disorder or neurodegenerative disease, such as PSP, e.g., at least in part, by detecting tau protein deposits (e.g., 4-repeat tau protein deposits), detecting of atrophy of the midbrain and/or superior cerebellar peduncles (e.g., using any of the imaging techniques described herein or known in the art, e.g., magnetic resonance imaging (MRI) or positron emission tomography (PET) scans), and/or detecting of hypometabolism in the frontal cortex, caudate, and/or thalamus in the subject (e.g., using any of the imaging techniques described herein or known in the art, e.g., MRI, CT scan, or PET scan).

In some embodiments, a subject can be identified as being at increased risk of developing a neurological disorder or neurodegenerative disease, such as PSP or identified as having a neurological disorder or neurodegenerative disease, such as PSP, e.g., at least in part, by detecting the presence of, or an elevated level (e.g., as compared to a level in a healthy control subject) of, one or more biomarkers in a subject.

In some embodiments, a subject can be identified as being at increased risk of developing a neurological disorder or neurodegenerative disease, by having had concussions/brain trauma as a result of sport activities (e.g. boxing, American football), military blast injuries or traffic accidents. In some embodiments, a subject can be identified as being at increased risk of developing a neurological disorder or neurodegenerative disease, by having reached an age over 65 years.

In some embodiments, a subject can be identified as being at increased risk of developing a neurological disorder or neurodegenerative disease, by having predisposing diseases such as heart disease, stroke, hypercholesterolemia, obesity, hypertension and diabetes.

The term “solvates” of the compounds is taken to mean adductions of inert solvent molecules onto the compounds, which are formed owing to their mutual attractive force. Solvates are, for example, mono- or dihydrates or alkoxides. The invention also comprises solvates of salts of the compounds according to the invention.

The compound of formula (I) and/or its tautomers are preferably used in its non-racemic form, i.e. as enantiomerically pure compound or its enantiomerically enriched mixture of the enantiomers. Most preferably, an enantiomerically enriched mixture denotes the compound of Formula (I) and/or its tautomers having an enantiomeric excess of more than 95% or more than 98%.

Desirably, the compositions of the invention are substantially free of corresponding isomers such as enantiomers of the compound of formula (I) and/or its tautomers. In one aspect, substantially free of corresponding isomers means at least 90% by weight of the compound of formula (I) and/or its tautomers to 10% by weight or less of a corresponding isomer in said pharmaceutical composition. In another aspect, substantially free of corresponding isomers means at least 95% by weight of the compound of formula (I) and/or its tautomers to 5% by weight or less of a corresponding isomer in the pharmaceutical composition. In yet another aspect, substantially free of corresponding isomers means at least 99% by weight of the compound of formula (I) and/or its tautomers to 1% by weight or less of a corresponding isomer in the pharmaceutical composition.

The compound of Formula (I) and/or its tautomers can be used in their final non-salt form. On the other hand, the present invention also encompasses the use of the compound (I) and/or its tautomers in the form of their pharmaceutically acceptable acid-addition salts, which can be formed by treating the respective compound with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoarylsulfonates, such as methanesulfonate, ethanesulfonate, toluenesulfonate and benzenesulfonate, and other organic acids and corresponding salts thereof, such as carbonate, acetate, trifluoroacetate, tartrate, maleate, succinate, citrate, benzoate, salicylate, ascorbate and the like. Accordingly, pharmaceutically acceptable acid-addition salts of the compounds according to the invention include the following: acetate, adipate, alginate, arginate, aspartate, benzoate, benzenesulfonate (besylate), bisulfate, bisulfite, bromide, butyrate, camphorate, camphorsulfonate, caprate, caprylate, chloride, chlorobenzoate, citrate, cyclamate, cinnamate, cyclopentanepropionate, digluconate, dihydrogenphosphate, dinitrobenzoate, dodecylsulfate, ethanesulfonate, formate, glycolate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this list is not intending to represent a restriction.

Acid addition salts of hydrochloric acid, maleic acid, tartraic acid, sulfuric acid or p-toluolsulfonic acid with compounds of formula (I) are especially preferred. The hydrochloric acid salt of formula (I) is most preferred.

A further aspect of the invention relates to the use of the medicaments and pharmaceutical compositions comprising the compound of formula (I) and/or its tautomers, and the respective dosage regimens for its administration to humans for inhibiting a glycosidase. Such use may be therapeutic or non-therapeutic in character. The term “inhibition” denotes any reduction in glycosidase activity, which is based on the action of the specific inventive compounds capable to interact with the target glycosidase in such a manner that makes recognition, binding and blocking possible.

In a preferred embodiment of the present invention, the glycosidase comprises glycoside hydrolases, more preferably family 84 glycoside hydrolases, most preferably (protein)-3-O—(N-acetyl-D-glucosaminyl)-L-serine/threonine N-acetylglucosaminyl hydrolase (O-GlcNAcase), highly preferably a mammalian O-GlcNAcase. It is particularly preferred that the compounds of formula (I) according to the invention selectively bind an O-GlcNAcase, e.g. thereby selectively inhibiting the cleavage of 2-acetamido-2-deoxy-β-D-glucopyranoside (O-GlcNAc) while they do not substantially inhibit a lysosomal β-hexosaminidase.

As discussed herein, the glycosidase-signaling pathways are relevant for various diseases, preferably neurodegenerative diseases, diabetes, cancer, cardiovascular diseases and stroke. Accordingly, the compounds according to the invention are useful in the prophylaxis and/or treatment of diseases that are dependent on the said signaling pathways by interaction with one or more of them. The present invention therefore relates to the therapeutic and non-therapeutic use of compounds according to the invention as inhibitors of the signaling pathways described herein, preferably of the OGA-mediated signaling.

A further aspect of the invention relates to a medicament comprising the compound of formula (I) and/or its tautomers and/or pharmaceutically usable salts, solvates and thereof. A “medicament” in the meaning of the invention is any agent in the field of medicine, which comprises the compound of formula (I) or preparations thereof (e.g. a pharmaceutical composition or pharmaceutical formulation) and can be used in prophylaxis, therapy, follow-up or aftercare of patients who suffer from diseases, which are associated with OGA activity, in such a way that a pathogenic modification of their overall condition or of the condition of particular regions of the organism could establish at least temporarily. The medicament is preferably prepared in a non-chemical manner, e.g. by combining the active ingredient with at least one solid, fluid and/or semi-fluid carrier or excipient, and optionally in conjunction with a single or more other active substances in an appropriate dosage form.

In the meaning of the invention, an “adjuvant” denotes every substance that enables, intensifies or modifies a specific response against the active ingredient of the invention if administered simultaneously, contemporarily or sequentially. Known adjuvants for injection solutions are, for example, aluminum compositions, such as aluminum hydroxide or aluminum phosphate, saponins, such as QS21, muramyldipeptide or muramyltripeptide, proteins, such as gamma-interferon or TNF, M59, squalen or polyols.

In particular, the present invention provides dosing regiments for the administration of the medicament or the pharmaceutical composition according to the present invention to human patients in need thereof. The dose regimens are especially suited for the treatment of neurological or neurodegenerative diseases including tauopathies such as PSP, Alzheimer's Disease or dementia.

Concerning O-GlcNAcase inhibition as therapeutic mechanism, a high degree of enzyme or target occupancy and/or inhibition over 24 hours is preferred. In this respect, maintaining sufficient O-GlcNAcase inhibitor concentrations at trough is a significant determinant to ensure minimal recovery of enzyme activity during treatment.

Hence, therapeutically effective doses of the compound of formula (I) and/or its tautomers preferably ensure target occupancies of at least about 35% at trough, preferably at least about 40% or 50% at trough, more preferably at least about 60%, or 70% at trough, most preferably at least about 80%, 90% or 95% at trough.

In one embodiment, the range of target occupancy at trough is about 40% to about 95%. In a further embodiment, the target occupancy at trough is about 60% to about 95%. In a further embodiment, the target occupancy at trough is at least about 70% to about 95%. In a further embodiment, the target occupancy at trough is at least about 80% to about 95%.

Therapeutically effective doses of the compound of formula (I) and/or its tautomers preferably ensure average target occupancies over a period of 24 hours during treatment with the medicament or formulation comprising the compound of formula (I), of at least about 70%, preferably at least about 80% or 85%, more preferably at least about 90%, most preferably at least about 95%.

In one embodiment, the range of average target occupancy over a period of 24 hours during treatment with the medicament or formulation comprising the compound of formula (I) and/or its tautomers or pharmaceutically usable solvates or salts thereof, is about 80% to about 95%. In a further embodiment, the average target occupancy over a period of 24 hours during treatment with the medicament or formulation comprising the compound of formula (I) and/or its tautomers or pharmaceutically usable solvates or salts thereof, is about 85% to about 98%. In a further embodiment, the average target occupancy over a period of 24 hours during treatment with the medicament or formulation comprising the compound of formula (I) and/or its tautomers or pharmaceutically usable solvates or salts thereof, is about 88% to about 99%.

Preferably, therapeutically effective doses ensure at least about 70% target occupancy in the brain at trough, and at least about 80% average target occupancy over a period of 24 hours during treatment with the medicament or formulation comprising the compound of formula (I) and/or its tautomers or pharmaceutically usable solvates or salts thereof.

Preferably, therapeutically effective doses ensure at least about 80% target occupancy in the brain at trough, and at least about 90% average target occupancy over a period of 24 hours during treatment with the medicament or formulation comprising the compound of formula (I) and/or its tautomers or pharmaceutically usable solvates or salts thereof.

In preferred embodiments target occupancy leads to clinical efficacy, that results in reduced or ameliorated progression of the signs and symptoms of a disease including neurological disorders or neurodegenerative diseases, diabetes, cancer, cardiovascular diseases and stroke and preferably a neurological disorder or neurodegenerative disease such as PSP. In one embodiment, the dose regimen comprising the compound of formula (I) and/or its tautomers reduces or ameliorate the progression of the signs and symptoms of a neurological disorder or neurodegenerative disease, such as PSP, in particular brain tau burden, whole brain and regional midbrain atrophy, reduced ability to perform daily activities, cognitive impairment. Moreover, the dose regimen comprising the compound of formula (I) and/or its tautomers improve biomarker results, overall clinical status and quality of life.

In one embodiment, the dose regimen comprising the compound of formula (I) and/or its tautomers reduces neuroinflammation. In one embodiment, the dose regimen comprising the compound of formula (I) and/or its tautomers is neuroprotective. In another embodiment, the dose regimen comprising the compound of formula (I) and/or its tautomers treats neuronal degeneration. In another embodiment, the dose regimen comprising the compound of formula (I) and/or its tautomers reduces atrophy or degeneration of the brain. In a further embodiment, the dose regimen comprising the compound of formula (I) and/or its tautomers reduces atrophy or degradation of the substantia nigra, globus pallidus, subthalamic nucleus and or cerebellum. In another embodiment, the dose regimen comprising the compound of formula (I) and/or its tautomers improves mitochondrial function. This results in improved behavior, improved survival (i.e. lifespan) and reduced brain degeneration. In another embodiment, the dose regimen comprising the compound of formula (I) and/or its tautomers reduces the aggregation of abnormal Tau protein, or fragments of Tau protein. In a further embodiment, the dose regimen comprising the compound of formula (I) and/or its tautomers ameliorates the reduction of neuroprotective proteins in the brain. In yet a further embodiment, the dose regimen comprising the compound of formula (I) and/or its tautomers reduces the reduction of brain-derived neurotrophic factor and Bcl 2. In a further embodiment, the dose regimen comprising the compound of formula (I) and/or its tautomers ameliorates the reduction of cerebral glucose metabolism as e.g. assessed by [¹⁸F]-fluoro-deoxyglucose PET.

Moreover, in preferred embodiments, the dose regimen comprising the compound of formula (I) and/or its tautomers results in improvement of one or more of the following disease parameters, biomarkers and scores compared to baseline values:
PSP Rating Scale (PSPRS) (28-item scale)
Modified PSPRS (10-item subscale)
Cortical Basal ganglionic Functional Scale (CBFS)
PSP Functional Disability Scale (PSPFDS)
Schwab and England Activities of Daily Living Scale (SEADL)
Clinical Global Impression of Change (CGI-C)
Progressive Supranuclear Palsy Quality of Life Scale (PSP-QoL)
Montreal Cognitive Assessment (MoCA)
Dimensional Apathy Scale (DAS)
Color Trails Test Parts 1 and 2 (CTT-1 and CTT-2)
Letter Fluency Test
Neurodegeneration and neuroinflammation CSF biomarkers, such as neurodegeneration panel: total tau, p-tau, NfL and neuroinflammation panel
Whole brain volumes as measured by volumetric brain MRI
Regional (midbrain, frontal lobes, third ventricle) volumes as measured by volumetric brain MRI
Plasma and CSF (NfL, total tau, p-tau) concentrations

In order to meet the required target occupancies in the brain established above, certain concentrations of the compound of formula (I) and/or its tautomers in the plasma need to be achieved in a human patient. The brain enzyme, receptor or target occupancy was quantified by competitive displacement of a selective radiolabelled O-GlcNAcase inhibitor used as positron emission tomography (PET) tracer by compound of formula (I). [¹⁸F]-LSN3316612 has been chosen as [¹⁸F]-radiolabelled, selective O-GlcNAcase inhibitor as it has been shown to exhibit a suitable selectivity and pharmacokinetics for quantification of O-GlcNAcase enzyme in the brain in preclinical studies and as a PET tracer for human studies (Paul S. et al. J. Nucl. Med. 2019, 60(1), 129-134). Other suitable PET tracers provide similar results. A correlation of the required target occupancies with plasma concentrations of the drug is established by measuring the plasma concentration of the compound of formula (I) and/or its tautomers during competitive displacement of the PET tracer by compound of formula (I) and/or its tautomers.

Thus, it has been found that the preferred target occupancies in the brain can be achieved when the plasma concentrations of the compound of formula (I) and/or its tautomers are as mentioned below. The plasma concentrations, if not indicated otherwise, are measured and indicated at steady state.

Throughout the specification, all embodiments of the present invention can be combined with any other embodiments of the invention disclosed in this specification, unless explicitly excluded. Generally, such combined embodiments are the more preferred, the more preferred individual embodiments they contain.

In a preferred embodiment, the plasma concentration of the compound of formula (I) and/or its tautomers is least about 35 ng/mL at trough, preferably at last about 45 ng/mL or 55 ng/mL or 65 ng/mL or 80 ng/mL at trough, more preferably at least about 100 ng/mL, or at least about 125 ng/mL or 155 ng/mL or 195 ng/mL at trough, most preferably at least about 270 ng/mL, 335 ng/mL, 610 ng/mL, 755 ng/mL, 1290 ng/mL or 1600 n/mL at trough.

In a very preferred embodiment, the plasma concentration of the compound of formula (I) and/or its tautomers may be at least about 45 ng/mL at trough or preferably at last about 55 ng/mL or 80 ng/mL at trough, more preferably at least about 125 ng/mL, or 195 ng/mL at trough, most preferably at least about 335 ng/mL, 755 ng/mL or 1600 ng/mL at trough.

In a further preferred embodiment, the range of plasma concentration of the compound of formula (I) and/or its tautomers at trough is about 45 ng/mL to about 2000 ng/mL, preferably about 55 ng/mL to about 1600 ng/mL. In a further embodiment, the range of plasma concentration at trough is about 100 ng/mL to about 2000 ng/mL, preferably is about 125 ng/mL to about 1600 ng/mL. In a further embodiment, the range of plasma concentration at trough is about 155 ng/mL to about 2000 ng/mL, preferably is about 125 ng/mL to about 1600 ng/mL. In a further embodiment, the range of plasma concentration at trough is about 270 ng/mL to about 2000 ng/mL, preferably about 335 ng/mL to about 1600 ng/mL.

Average plasma concentrations of the compound of formula (I) and/or its tautomers over a period of 24 hours during treatment with the medicament or formulation comprising the compound of formula (I) at steady state, are preferably at least about 155 ng/mL, at least about 195 ng/mL or preferably at last about 270 ng/mL, 335 ng/mL, 380 ng/mL or 475 ng/mL, more preferably at least about 610 ng/mL or 750 ng/mL, most preferably at least about 1290 ng/mL or 1600 ng/mL.

In a very preferred embodiment, average plasma concentrations of the compound of formula (I) and/or its tautomers over a period of 24 hours during treatment with the medicament or formulation comprising the compound of formula (I), are at least about 195 ng/mL, preferably at last about 335 ng/mL or 475 ng/mL, more preferably at least about 750 ng/mL, most preferably at least about 1600 ng/mL.

In one embodiment, the range of average plasma concentrations of the compound of formula (I) and/or its tautomers over a period of 24 hours during treatment with the medicament or formulation comprising the compound of formula (I) and/or its tautomers or pharmaceutically usable solvates or salts thereof, is about 270 ng/mL to about 2000 ng/mL, preferably about 335 ng/mL to about 1600 ng/mL. In a further embodiment, the range of average plasma concentrations of the compound of formula (I) and/or its tautomers over a period of 24 hours during treatment with the medicament or formulation comprising the compound of formula (I) and/or its tautomers or pharmaceutically usable solvates or salts thereof, is about 380 ng/mL to about 5000 ng/mL preferably about 475 ng/mL to about 4120 ng/mL. In a further embodiment, the range of average plasma concentrations of the compound of formula (I) and/or its tautomers over a period of 24 hours during treatment with the medicament or formulation comprising the compound of formula (I) and/or its tautomers or pharmaceutically usable solvates or salts thereof, is about 490 ng/mL to about 10000 ng/mL, preferably about 615 ng/mL to about 8330 ng/mL.

In one embodiment, plasma concentrations of the compound of formula (I) and/or its tautomers at steady state are at least about 155 ng/mL or 195 ng/mL at trough, while average plasma concentrations of the compound of formula (I) and/or its tautomers over a period of 24 hours are at least about 270 ng/mL or 335 ng/mL.

In a very preferred embodiment, plasma concentrations of the compound of formula (I) and/or its tautomers at steady state are at least about 195 ng/mL at trough, while average plasma concentrations of the compound of formula (I) and/or its tautomers over a period of 24 hours are at least about 335 ng/mL.

In a further embodiment, plasma concentrations of the compound of formula (I) and/or its tautomers at steady state are at least about 270 ng/mL or 335 ng/mL at trough, while average plasma concentrations of the compound of formula (I) and/or its tautomers over a period of 24 hours are at least about 610 ng/mL or 755 ng/mL.

In a very preferred embodiment, plasma concentrations of the compound of formula (I) and/or its tautomers at steady state are at least about 335 ng/mL at trough, while average plasma concentrations of the compound of formula (I) and/or its tautomers over a period of 24 hours are at least about 755 ng/mL.

The present invention provides for a dose regimen for the administration of a medicament or a pharmaceutical composition comprising of formula (I) and/or its tautomers or pharmaceutically usable solvates or salts thereof to an individual in need thereof, for example, and individual having a neurological disorder or neurodegenerative disease, such as mild to moderate AD or PSP, so as to obtain a desired pharmacokinetic profile of a desired concentration of the compound of formula (I) and/or its tautomers in the plasma over a period of time. By maintaining a preferred plasma concentration of the compound of formula (I) and/or its tautomers, a preferred target occupancy is achieved.

Preferred pharmacokinetic profiles and/or endpoints may preferably be achieved through the administration of one or more unit dosage forms comprising, for example, about 100 mg, about 120 mg, about 150 mg, about 180 mg, about 200 mg, about 250 mg, about 300 mg, about 350 mg, about 375 mg, about 400 mg, about 450 mg, about 500 mg or about 750 mg of the compound of formula (I) and/or its tautomers, which may also be administered in the form of a pharmaceutically useable solvate or salt thereof.

Preferred pharmacokinetic profiles and/or endpoints may more specifically be achieved through the administration of specific daily doses of the compound of formula (I) and/or its tautomers, preferably daily oral doses, preferably ranging from about 240 mg to about 1500 mg per day, more preferably about 300 mg to about 1200 mg per day and most preferably about 360 mg to about 1000 mg per day. In a further preferred embodiment, the daily dose is about 300 mg or about 450 mg or about 500 mg or about 600 mg or about 900 mg or about 1000 mg per day.

In preferred embodiments, the daily dose of the compound of formula (I) and/or its tautomers is achieved by a BID or TID dose regimen, which comprise the BID or TID administration of the respective portion of the daily dose of the compound of formula (I) and/or its tautomers. This is preferably achieved by administration of one or two unit dosage forms comprising of the compound of formula (I) and/or its tautomers, such as a tablet or capsule per administration.

In a preferred embodiment, wherein BID or TID dose regimens are applied, immediate release formulations of the compound of formula (I) and/or its tautomers are preferred. In a further embodiment, wherein once daily dosing (QD) of the compound of formula (I) and/or its tautomers is applied, modified, controlled or slow or sustained release formulations are preferred, and more preferably formulations that release the dose in a constant manner, i.e. without large “burst”-effect, where a large part of the dose is released at once.

Immediate release formulations and immediate release unit dosage forms according to the present invention are designed to release the compound of formula (I) and/or its tautomers or pharmaceutically usable solvates or salts thereof, immediately after the outer shell such as a coating of the respective dosage form, such as a tablet or a capsule dissolves. This preferably results in rapid absorption and fast systemic entry into the body, i.e. a prompt increase in blood concentration of the compound of formula (I) and/or its tautomers. Immediate release formulations and immediate release unit dosage forms according to the present invention also comprise a sachet or a sachet formulation.

Preferably, the immediate release dosage form according to the invention has released at least 75%, preferably at least 95% of the compound of formula (I) and/or its tautomers at 45 minutes and/or at least 90% of the compound of formula (I) and/or its tautomers at 15 minutes in the USP Paddle test, preferably in 0.1% cetyltrimethylammonium bromide (CTAB) in 0.01 M hydrochloric acid as dissolution medium, and paddle speed of 75 rpm. Most preferably, the immediate release dosage form according to the invention has released at least 75% of the compound of formula (I) and/or its tautomers at 45 minutes in the USP Paddle test as described in example 4.

In very preferred embodiments, the dose regimen of the medicament or formulation comprising the compound of formula (I) and/or its tautomers or pharmaceutically usable solvates or salts thereof comprises a respective dose and a dosing frequency as follows: A dose the compound of formula (I) and/or its tautomers of about 150 mg thrice a day (TID) or about 250 mg twice a day (BID), or preferably about 300 mg thrice a day or 500 mg of the compound of formula (I) twice a day.

In the case where individual patients could not tolerate the preferred doses and dose regimens disclosed in this specification, clinical beneficially effects on the respective indications can still be seen with a low dose and a low dose regimen. This low dose and low dose regimen is more preferably specifically disclosed in embodiments A-F.
A. The compound and/or its tautomers for use in a method of treating a human subject, the method comprising the repeated administration of one or more unit dosage forms comprising the compound of formula (I) and/or its tautomers in a dose and at a daily dosing frequency, wherein the dose of the compound of formula (I) and/or its tautomers is orally administered and is in the range of about 75 mg to about 250 mg.
B. The compound and/or its tautomers for use according to embodiment A, wherein the dose of the compound of formula (I) and/or its tautomers is orally administered and is in the range of about 125 mg to about 250 mg.
C. The compound and/or its tautomers for use according to embodiment A or B, wherein the repeatedly administered dose of the compound of formula (I) and/or its tautomers remains constant.
D. The compound and/or its tautomers for use according to embodiments A to C, wherein the daily dosing frequency of the compound of formula (I) and/or its tautomers is twice a day or thrice a day.
E. The compound and/or its tautomers for use according to any one of the previous embodiments A to D, wherein the daily dose of the compound of formula (I) and/or its tautomers is in the range of about 225 mg to 250 mg per day, when orally administered.
F. The compound and/or its tautomers for use according to any one of the previous embodiments A to E, comprising the administration of an oral dose of the compound of formula (I) and/or its tautomers of about 75 mg thrice a day or about 125 mg twice a day.

In one embodiment, the invention provides for a medicament comprising the compound of formula (I) and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers of about 1650 to about 7390 ng/mL. In a further embodiment, the plasma C max of the compound of formula (I) and/or its tautomers is preferably from about 1940 to about 6750 ng/mL. In another more preferred embodiment, the C max of the compound of formula (I) and/or its tautomers is about 2050 ng/mL to about 2160 ng/mL or about 2200 ng/mL to about 2950 ng/mL or about 3030 ng/mL to about 3150 ng/mL or about 4250 ng/mL to about 4350 ng/mL or about 4400 ng/mL to about 6000 ng/mL or about 6130 ng/mL to about 6250 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers about 1650 to about 3520 ng/mL. In a further embodiment, the plasma C max of the compound of formula (I) and/or its tautomers is preferably from about 1940 to about 3340 ng/mL. In another more preferred embodiment, the C max is about 2050 ng/mL to about 2160 ng/mL or about 2200 ng/mL to about 2950 ng/mL or about 3030 ng/mL to about 3150 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers about 1650 to about 2930 ng/mL. In a further embodiment, the plasma C max of the compound of formula (I) and/or its tautomers is preferably from about 1940 to about 2380 ng/mL. In another more preferred embodiment, the C max of the compound of formula (I) and/or its tautomers is about 2050 ng/mL or about 2100 ng/mL or about 2160 ng/mL or about 2200 ng/mL or about 2250 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers of about 2500 to about 3520 ng/mL. In a further embodiment, the plasma C max of the compound of formula (I) and/or its tautomers is preferably from about 2700 to about 3340 ng/mL. In another more preferred embodiment, the C max of the compound of formula (I) and/or its tautomers is about 2950 ng/mL or about 3000 ng/mL or about 3030 ng/mL or about 3100 ng/mL or about 3150 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers of about 3580 to about 7390 ng/mL. In a further embodiment, the plasma C max of the compound of formula (I) and/or its tautomers is preferably from about 3910 to about 6750 ng/mL. In another more preferred embodiment, the C max of the compound of formula (I) and/or its tautomers is about 4250 ng/mL to about 4350 ng/mL or about 4400 ng/mL to about 6000 ng/mL or about 6130 ng/mL to about 6250 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers of about 3580 to about 5600 ng/mL. In a further embodiment, the plasma C max of the compound of formula (I) and/or its tautomers is preferably from about 3910 to about 4790 ng/mL. In another more preferred embodiment, the C max of the compound of formula (I) and/or its tautomers is about 4250 ng/mL or about 4300 ng/mL or about 4350 ng/mL or about 4400 ng/mL or about 4450 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma C max of the compound of formula (I) and/or its tautomers of about 5150 to about 7390 ng/mL. In a further embodiment, the plasma C max of the compound of formula (I) and/or its tautomers is preferably from about 5510 to about 6750 ng/mL. In another more preferred embodiment, the C max of the compound of formula (I) and/or its tautomers is about 6000 ng/mL or about 6100 ng/mL or about 6130 ng/mL or about 6200 ng/mL or about 6250 ng/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 13850 to about 90500 ng*h/mL. In a further embodiment, the plasma AUC over 24 hours of the compound of formula (I) and/or its tautomers is preferably from about 18550 to about 63220 ng*h/mL. In another more preferred embodiment, the AUC over 24 hours of the compound of formula (I) and/or its tautomers is about 20000 ng*h/mL to about 23200 ng*h/mL or about 25750 ng*h/mL to about 29000 ng*h/mL or about 43000 ng*h/mL to about 46780 ng*h/mL or about 52680 ng*h/mL to about 55000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 13850 to about 40365 ng*h/mL. In a further embodiment, the plasma AUC over 24 hours of the compound of formula (I) and/or its tautomers is preferably from about 18550 to about 30900 ng*h/mL. In another more preferred embodiment, the AUC over 24 hours of the compound of formula (I) and/or its tautomers is about 20000 ng*h/mL to about 23200 ng*h/mL or about 25750 ng*h/mL to about 29000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 13850 to about 39800 ng*h/mL. In a further embodiment, the plasma AUC over 24 hours of the compound of formula (I) and/or its tautomers is preferably from about 18550 to about 27850 ng*h/mL. In another more preferred embodiment, the AUC over 24 hours of the compound of formula (I) and/or its tautomers is about 20000 ng*h/mL or about 21000 ng*h/mL or about 23200 ng*h/mL or about 25000 ng*h/mL or about 26000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its is provided in an amount sufficient to result in a plasma AUC over 24 hours of the compound of formula (I) and/or its tautomers of about 15990 to about 40365 ng*h/mL. In a further embodiment, the plasma AUC over 24 hours of the compound of formula (I) and/or its tautomers is preferably from about 20600 to about 30900 ng*h/mL. In another more preferred embodiment, the AUC over 24 hours of the compound of formula (I) and/or its tautomers is about 22000 ng*h/mL or about 23000 ng*h/mL or about 25750 ng*h/mL or about 28000 ng*h/mL or about 29000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 30150 to about 90500 ng*h/mL. In a further embodiment, the plasma AUC over 24 hours of the compound of formula (I) and/or its tautomers is preferably from about 37420 to about 63220 ng*h/mL. In another more preferred embodiment, the AUC over 24 hours of the compound of formula (I) and/or its tautomers is about 43000 ng*h/mL to about 46780 ng*h/mL or about 52680 ng*h/mL to about 55000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 30150 to about 74230 ng*h/mL. In a further embodiment, the plasma AUC over 24 hours of the compound of formula (I) and/or its tautomers is preferably from about 37420 to about 56130 ng*h/mL. In another more preferred embodiment, the AUC over 24 hours of the compound of formula (I) and/or its tautomers is about 43000 ng*h/mL or about 44000 ng*h/mL or about 46780 ng*h/mL or about 49000 ng*h/mL or about 50000 ng*h/mL.

In one embodiment, the invention provides for a medicament comprising the compound of formula (I) and/or its tautomers or pharmaceutically usable solvates or salts thereof and a method of administering a medicament or a pharmaceutical composition comprising the compound of formula (I) and/or its tautomers or pharmaceutically usable solvates or salts thereof to an individual, wherein said compound of formula (I) and/or its tautomers is provided in an amount sufficient to result in a plasma AUC over 24 hours of the compound of formula (I) and/or its tautomers of about 30750 to about 90500 ng*h/mL. In a further embodiment, the plasma AUC over 24 hours of the compound of formula (I) and/or its tautomers is preferably from about 42140 to about 63220 ng*h/mL. In another more preferred embodiment, the AUC over 24 hours of the compound of formula (I) and/or its tautomers is about 49000 ng*h/mL or about 50000 ng*h/mL or about 52680 ng*h/mL or about 54000 ng*h/mL or about 55000 ng*h/mL.

The time to achieve plasma C max of the compound of formula (I) and/or its tautomers will depend upon the individual to be treated, but is preferably between 0.5 to 6 hours. In various preferred embodiments, the t max (time to C max) is from about 0.75 to 2 hours, or is from about 1.00 hour to about 1.75 hours, or is about 1.5 hours. Preferably, t max is ranging from 1 to 1.5 hours after administration.

Further, the invention encompasses repeated dosing of the compound of formula (I) and/or its tautomers to achieve these levels for about 1 week, two weeks, three weeks, four weeks, one month, two months, three months, four months, five months, six months, seven months, eight months, nine months, ten months, eleven months, one year, or preferably more than one year.

All dosages given throughout the specification are referring to the amount of the compound of formula (I) and/or its tautomers, in its respective free base form. Thus, any dose or dosage of an acid-addition salt of the compound of formula (I) and/or its tautomers is to be adapted accordingly, taking the additional molecular weight of the respective acid into account. The same applies correspondingly to solvates of the compound of formula (I) and/or its tautomers.

It is understood that while the compound of formula (I) and/or its tautomers may be administered in form of one or more respective pharmaceutically usable solvate or salt, plasma concentrations are given throughout the specification in the respective base form, i.e. as free compound of formula (I) and/or its tautomers.

In preferred embodiments the compound of formula (I) and/or its tautomers are administered in form of the respective hydrochloride.

Preferred intervals between administrations are regular or follow a regular pattern. More preferably, the medicament of the present invention is administered 2 or 3 times daily. Most preferably, the interval between administrations is about 4 to about 10 or to about 12 hours for twice daily administrations and about 4 to about 6 hours, for thrice daily administration during the day, followed by a period of non-dosing during night times for e.g. 8 to 16 hours.

A low dose regimen provides the compound of formula (I) and/or its tautomers to the individual in a daily dose of about 450 mg or about 500 mg. A low dose regimen can, for example, be used before or after a dosing regimen with a higher dose, such as a daily dose of about 900 mg or about 1000 mg.

Oral administration of a dose of the compound of formula (I) and/or its tautomers, preferably twice or thrice daily for at least about 4 months, preferably at least about 6 or at least about 8 months, and more preferably at least about 1 year, or at least about 2 years provides an improvement or lessening of decline in cognitive function, biochemical disease marker progression, and/or plaque pathology.

In one aspect, a preferred dosage form is a unit dosage form, such as a tablet. In another aspect, a preferred dosage form is a capsule. In another aspect, a preferred dosage form is a powder, preferably contained in a sachet. In other aspects, the medicament or composition provides an improvement or lessening in decline in biochemical disease marker progression, plaque pathology, quality of life indicators or combinations of any disease parameters.

The decline in cognitive function preferably can be characterized by cognition tests. It is preferred that the lessening in decline in cognitive function is at least 25% as compared to individuals treated with placebo, more preferably at least 40%, and even more preferably at least 60%. For example, an individual treated with placebo having probably mild-to-moderate Alzheimer's disease is expected to score approximately 5.5 points higher on the ADAS-cog test after a specified period of time (e.g. 1 year) whereas an individual treated with a composition of the invention for the same period of time will score only approximately 3.3 points higher on the ADAS-cog scale, i.e., will show 60% of the decline in cognitive function relative to untreated individuals, or 2.2 points higher i.e., will show 40% of the decline in cognitive function relative to untreated individuals, when treated for the same specified period of time.

In a specific embodiment of this aspect of the invention, the dosage is provided as a medicament or a pharmaceutical composition that is composed of the compound of formula (I) and/or its tautomers or a pharmaceutically acceptable salt thereof, an optional release agent, and additional optional ingredients.

In another specific embodiment of this aspect of the invention, the dosage is provided as a medicament or pharmaceutical composition that is a unit dosage form preferably a tablet or a capsule. The unit dosage form is preferably composed of the compound of formula (I) and/or its tautomers, microcrystalline cellulose, colloidal silicon dioxide, and magnesium stearate. In another specific embodiment of this aspect of the invention, the dosage is provided as a pharmaceutical composition that is a capsule is composed of the compound of formula (I), microcrystalline cellulose, colloidal silicon dioxide, and magnesium stearate, all encapsulated in lactose monohydrate, hydroxyl propyl methyl cellulose, titanium dioxide, tracetin/glycerol triacetate, and iron oxide.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using processes known in the pharmaceutical art by, e.g., combining the active ingredient with the excipient(s) or adjuvant(s).

The pharmaceutical composition of the invention is produced in a known way using common solid or liquid carriers, diluents and/or additives and usual adjuvants for pharmaceutical engineering and with an appropriate dosage. The amount of excipient material that is combined with the active ingredient to produce a single dosage form varies depending upon the host treated and the particular mode of administration. Suitable excipients include organic or inorganic substances that are suitable for the different routes of administration, such as enteral (e.g. oral), parenteral or topical application, and which do not react with compounds of formula (I) or salts thereof.

Examples of suitable excipients are water, vegetable oils, benzyl alcohols, alkylene glycols, polyethylene glycols, glycerol triacetate, gelatin, carbohydrates, e.g. lactose or starch, magnesium stearate, talc and petroleum jelly.

In a very preferred embodiment of the present invention, the pharmaceutical composition is adapted for oral administration. The preparations can be sterilized and/or can comprise auxiliaries, such as carrier proteins (e.g. serum albumin), lubricants, preservatives, stabilizers, fillers, chelating agents, antioxidants, solvents, bonding agents, suspending agents, wetting agents, emulsifiers, salts (for influencing the osmotic pressure), buffer substances, colorants, flavorings and one or more further active substances, for example one or more vitamins. Additives are well known in the art, and they are used in a variety of formulations.

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

It goes without saying that, in addition to the above particularly mentioned constituents, the formulations may also comprise other agents usual in the art with respect to the particular type of formulation; thus, for example, formulations which are suitable for oral administration may comprise flavors.

A preferred example of a capsule or tablet core formulation according to the invention comprises the compound of formula (I) and/or its tautomers, preferably in a salt form, such as the hydrochloride, a filler, a binder, a disintegrant, a glidant, a dry binder, a lubricant and optionally a solvent.

A very preferred example of a capsule or tablet core formulation comprises the compound of formula (I) and/or its tautomers, preferably in a salt form, such as the hydrochloride, microcrystalline cellulose, povidone, croscarmellose sodium, silica, copovidone and sodium stearyl fumarate and optionally a solvent, such as water.

Furthermore, the pharmaceutical compositions and medicaments according to the invention may be administered alone or in combination with other treatments. A synergistic effect may be achieved by using more than one active pharmaceutical ingredient in the pharmaceutical composition or medicament, i.e. in this case, the compound of formula (I) and/or its tautomers is combined with at least another agent as active ingredient. The active ingredients can be used either simultaneously or sequentially and can be formulated into a single unit dosage form comprising both, the compound of formula (I) and a further pharmaceutically active ingredient or agent. The present compounds are suitable for combination with agents known to those of skill in the art (e.g., WO 2008/025170) and are useful with the pharmaceutical compositions and medicaments according to the invention.

In some embodiments, pharmaceutical compositions and medicaments according to the invention, and the dose regimens according to the invention, may be provided in combination with any other active agents or pharmaceutical compositions where such combined therapy may be useful to modulate O-GlcNAcase activity, for example to treat neurodegenerative, inflammatory, cardiovascular, or immunoregulatory diseases or any condition described herein. In some embodiments, pharmaceutical compositions and medicaments according to the invention, and the dose regimens according to the invention, may be provided in combination with one or more agents useful in the prevention or treatment of tauopathies, synucleinopathies and Alzheimer's disease. Examples of such agents may include, without limitation,
Acetylcholine esterase inhibitors (AChEIs) such as Aricept® (Donepezil), Exelon® (Rivastigmine), Razadyne® (Razadyne ER®, Reminyl®, Nivalin®, Galantamine), Cognex® (Tacrine), NMDA antagonists such as memantine (Axura®, Ebixa®), Huperzine A, Phenserine, Debio-9902 SR (ZT-1 SR), Zanapezil (TAK0147), ganstigmine, NP7557, α7 nicotinic acetylcholine receptor agonists, 5-HT6 receptor antagonists, M1 muscarinic acetylcholine receptor agonists and positive allosteric modulators, and other agents which restore/potentiate cholinergic signaling or show pro-cognitive effects
Tau aggregation inhibitors such as methylene blue, morphomers, and others
Agents blocking intra- and extracellular tau seeding and transcellular propagation by sequestration of tau by directly binding to tau such as tau antibodies and other protein/peptide derived tau binding agents
Vaccines inducing a titer of tau binding/sequestering antibodies
Microtubule stabilizers such as AL-108, AL-208, paclitaxel and others
Neuroprotective agents or claimed as such (e.g. AZP2006)
Anti-inflammatory agents such as non-steroidal anti-inflammatory drugs, TNFα/anti-rheumatic drugs such as Enbrel, Humira and others
siRNAs, shRNAs, gene therapies or CRISPER derived therapeutic agents that reduce or modify the expression of tau and/or downregulate the generation of Aβ
Amyloid-μ (Aβ) peptide lowering agents such as β-secretase (BACE-1) or γ-secretase inhibitors or modulators
Senile plaque-clearing, As monomer or oligomer binding biologics such as Aβ antibodies and Aβ vaccines inducing a titer of such antibodies
Compounds that inhibit tau phosphorylation by inhibiting kinases such as GSK3 inhibitors (Tideglusib)
TREM2 and CD33 binding/modulating drugs/antibodies/vaccines
Cholesterol lowering drugs such as Zocor/Lipitor, ApoE4 targeting drugs
Anti-hypertensive drugs such a diuretics, angiotensin-1 receptor blockers, angiotensin-converting enzyme inhibitors, calcium channel blockers, or β-blockers
Mood stabilizing drugs such as anti-depressants (e.g. fluoxetine, duloxetine, bupropion, amitriptyline, imipramine)
Insomnia drugs such as Zolpidem
Dietary supplements such as Coenzyme Q10
Dopaminergic drugs such as Carbiodopa/Levodopa, monoaminooxidase inhibitors (Razagiline), COMPT inhibitors, gene therapies and others
Lewy body/α-synuclein binding biologics such as α-synuclein antibodies and α-synuclein vaccines inducing a titer of such antibodies
TDP43 or FUS binding biologics such as TDP43 or FUS antibodies and TDP43 or FUS vaccines inducing a titer of such antibodies
Progranulin modulating agents
Cell-based therapies to enhance synaptic plasticity and/or neuronal networks with the aim to improve cognition
Deep brain stimulation (DBS) consist of implanting electrodes in key brain areas, but without limitation, such as the fornix and/or the nucleus basalis of Meynert with the aim to stimulate neuronal pathways involved in memory and cognition. (Mirzadeh Z. et al. (2016) J. Neural Transm (Vienna) 123: 775-783; Ponce F A et al. (2016) J Neurosurg 125:75-84; Sankar T et al. (2015) Brain Stimulat 8:645-654; Lozano A M et al. (2016) J Alzheimers Dis 54:777-787; Kuhn J et al. (2015) Brain Stimulat 8:838-839)

In addition to the chronically applied pharmacological therapies listed above, there may be benefits to either short or long-term O-GlcNAcase inhibition in more invasive brain therapies including cell-based therapies, gene therapies, deep brain stimulation and targeted lesioning of brain circuits. For instance, cell transplantation of iPSC cells in diseases like Parkinson's disease may produce cellular stresses in the cultured cells that may be mitigated by treatment with an O-GlcNAcase inhibitor to improve function and survival concomitant to and after the procedure. As reviewed by Martinez et al (2017), O-GlcNAc has been implicated in mediating cell survival decisions via numerous pathways that include transcription, stress granule formation, Heat Shock Protein synthesis, altered metabolic flux, reduced endoplasmic reticulum (ER) stress, and improved mitochondrial function which all may play a role in determining the immediate and long-term function and survival of the transplanted cells. In addition, reduced cellular stresses in the operative site and reduction of subsequent neuroinflammatory responses may also be of benefit.

In some embodiments, pharmaceutical compositions and medicaments according to the invention, and the dose regimens according to the invention, may be provided in combination with one or more agents useful in the prevention or treatment of tauopathies, synucleinopathies and Alzheimer's disease. Examples of such agents may include, without limitation, cell replacement therapies which consist of generation of neuronal cells that are transplanted in any affected brain areas with the aim to improve cognition. The neuronal cells can be generated directly from somatic cells (Vierbuchen T. et al. (2010) Nature 463:1035-1041; Zhang S Z. et al. (2016) Stem Cells Int. 2016: 2452985; Addis R C. et al. (2011) PLoS ONE 6: e28719; Zhao J. et al. (2012) PLoS ONE 7: e41506; Lim M S. et al. (2015) J. Biol. Chem. 290:17401-17414) and/or from induced pluripotent stem cells (iPSCs) (Kim J. et al. (2011) Proc Natl Acad Sci USA 108:7838-7843; Matsui T. et al. (2012) Stem Cells 30: 1109-1119; Sheng C. et al. (2012) Cell Res 22: 208-218; Lujan E. et al. (2012) Proc Natl Acad Sci USA 109:2527-2532; Lim M S et al. (2015) J Biol Chem 290:17401-17414; Han D W et al. (2012) Cell Stem Cell 10: 465-472) by using any combination of neural-specific transcription factors.

The invention also relates to a set (kit) consisting of separate packs of an effective amount of the compound of formula (I) and/or pharmaceutically acceptable salts and solvates thereof and an effective amount of a further medicament comprising a pharmaceutically active ingredient. The set comprises suitable containers, such as boxes, individual bottles, bags or ampoules. The set may, for example, comprise separate unit dosage forms, each containing an effective amount of the compound of formula (I) and/or pharmaceutically acceptable salts and solvates thereof and an effective amount of a further medicament comprising a pharmaceutically active ingredient in further unit dosage forms as tablets or e.g. dissolved or in lyophilized form.

The pharmaceutical compositions and medicaments according to the invention can be administered before or following an onset of disease once or several times acting as therapy. The aforementioned pharmaceutical compositions and medicaments are particularly used for the therapeutic treatment. A therapeutically relevant effect relieves to some extent one or more symptoms of a disorder, or returns to normality, either partially or completely, one or more physiological or biochemical parameters associated with or causative of a disease or pathological condition. Monitoring is considered as a kind of treatment provided that the pharmaceutical compositions and medicaments are administered in distinct intervals, e.g. in order to booster the response and eradicate the symptoms of the disease completely.

In the meaning of the invention, prophylactic treatment is advisable if the subject possesses any preconditions for the aforementioned physiological or pathological conditions, such as a familial disposition, a genetic defect, or a previously passed disease.

Particularly preferred embodiments of the present invention are the following:

A. A compound of formula (I) and/or its tautomers for use in a method of treating a human subject, the method comprising the repeated administration of one or more unit dosage forms comprising the compound of formula (I) and/or its tautomers in a dose and at a daily dosing frequency sufficient to maintain the plasma concentration of the compound of formula (I) and/or its tautomers at steady state at at least about 35 ng/mL at trough.

B. The compound and/or its tautomers for use according to embodiment A, wherein the plasma concentration of the compound of formula (I) and/or its tautomers is maintained in a range of about 45 ng/mL to about 2000 ng/mL at trough.

C. A compound of formula (I) and/or its tautomers for use in a method of treating a human subject, the method comprising the repeated administration of one or more unit dosage forms comprising the compound of formula (I) and/or its tautomers in a dose and at a daily dosing frequency sufficient to maintain the average plasma concentration of the compound of formula (I) and/or its tautomers over a period of 24 hours at steady state at at least about 155 ng/mL.

D. The compound and/or its tautomers for use according to embodiment C, wherein the average plasma concentration of the compound of formula (I) and/or its tautomers is maintained over a period of 24 hours in a range of about 270 ng/mL to about 2000 ng/mL.

E. A compound of formula (I) and/or its tautomers for use in a method of treating a human subject, the method comprising the repeated administration of one or more unit dosage forms comprising the compound of formula (I) and/or its tautomers in a dose and at a daily dosing frequency sufficient to maintain at steady state a plasma C max of the compound of formula (I) and/or its tautomers of about 1650 to about 7390 ng/mL.

F. A compound of formula (I) and/or its tautomers for use in a method of treating a human subject, the method comprising the repeated administration of one or more unit dosage forms comprising the compound of formula (I) and/or its tautomers in a dose and at a daily dosing frequency sufficient to maintain at steady state a plasma AUC over 24 hours of the compound of formula (I) and/or its tautomers of about 13850 to about 90500 ng*h/mL.

G. The compound and/or its tautomers for use according to any one of the previous embodiments, wherein the dose of the compound of formula (I) and/or its tautomers is orally administered and is in the range of about 150 mg to about 500 mg.

H. The compound and/or its tautomers for use according to embodiment E, wherein the plasma C max of the compound of formula (I) and/or its tautomers is about 3580 to about 7390 ng/mL. 1. The compound and/or its tautomers for use according to embodiment F, wherein the plasma AUC over 24 hours of the compound of formula (I) and/or its tautomers is about 30150 to about 90500 ng*h/mL.

J. The compound and/or its tautomers for use according to any one of the previous embodiments, wherein the dose of the compound of formula (I) and/or its tautomers is orally administered and is in the range of about 300 mg to about 500 mg.

K. The compound and/or its tautomers for use according to any one of the previous embodiments, wherein the repeatedly administered dose of the compound of formula (I) and/or its tautomers remains constant.

L. The compound and/or its tautomers for use according to any one of the previous embodiments, wherein the daily dosing frequency of the compound of formula (I) and/or its tautomers is twice a day or thrice a day.

M. The compound and/or its tautomers for use according to any one of the previous embodiments, wherein the daily dose of the compound of formula (I) and/or its tautomers is about 240 mg to about 1500 mg per day, when orally administered.

N. The compound and/or its tautomers for use according to any one of the previous embodiments, comprising the administration of an oral dose of the compound of formula (I) and/or its tautomers of about 150 mg thrice a day or about 250 mg twice a day, or about 300 mg thrice a day or about 500 mg twice a day.

O. The compound and/or its tautomers for use according to any one of the previous embodiments, wherein the human subject suffers from a disease or condition, or is at increased risk of developing a disease or condition.

P. The compound and/or its tautomers for use according to embodiment 0, wherein the disease or condition is selected from one or more proteinopathies.

Q. The compound and/or its tautomers for use according to embodiment O or P, wherein the disease or condition is selected from neurological disorders or neurodegenerative diseases, diabetes, cancer, cardiovascular diseases and stroke.

R. A compound of formula (I) and/or its tautomers for use in a method of treating a human subject suffering from or being at increased risk of developing a disease or condition selected from neurological disorders or neurodegenerative diseases, diabetes, cancer, cardiovascular diseases and stroke, the method comprising the administration of an oral dose of the compound of formula (I) and/or its tautomer

of about 150 mg thrice a day or about 250 mg twice a day, or about 300 mg thrice a day or 500 mg twice a day.

S. The compound and/or its tautomers for use according to embodiment 0, P, Q or R, wherein the disease or condition is selected from the group of one or more tauopathies, synucleinopathies and Alzheimer's disease (AD), amyotrophic lateral sclerosis (ALS), amyotrophic lateral sclerosis with cognitive impairment (ALSci), argyrophilic grain disease, behavioral variant frontotemporal dementia (bvFTD), non-fluent and semantic variant primary progressive aphasia (nfv & svPPA), Bluit disease, corticobasal degeneration (CBD), Dementia pugilistica, Dementia with Lewy Bodies (DLB), diffuse neurofibrillary tangles with calcification, Down's syndrome, Familial British dementia, Familial Danish dementia, frontotemporal dementia with parkinsonism linked to chromosome 17 (FTDP-17), frontotemporal lobar degeneration (FTLD), ganglioglioma, gangliocytoma, Gerstmann-Straussler-Scheinker disease, globular glial tauopathy, Guadeloupean parkinsonism, Hallevorden-Spatz disease (neurodegeneration with brain iron accumulation type 1), lead encephalopathy, lipofuscinosis, meningioangiomatosis, multiple system atrophy (MSA), myotonic dystrophy, Niemann-Pick disease including Niemann-Pick disease type C, Pallido-ponto-nigral degeneration, Parkinson's disease, Parkinson's disease dementia (PDD), Parkinsonism-dementia complex of Guam, Pick's disease (PiD), postencephalitic parkinsonism (PEP), Prion diseases, including Creutzfeldt-Jakob Disease (GJD), and variant Creutzfeldt-Jakob Disease (vCJD), fatal Familial Insomnia, Kuru, progressive supercortical gliosis, progressive supranuclear palsy (PSP), pure autonomic failure, Richardson's syndrome, subacute sclerosing panencephalitis, Tangle-only dementia, tuberous sclerosis, Huntington's disease or mild cognitive impairment (MCI), Chronic traumatic encephalopathy, Primary progressive aphasia, Progressive nonfluent aphasia, Semantic dementia, Steele-Richardson-Olszewski syndrome, epilepsy, chronic and acute inflammation, Crohn disease, neuroinflammation, subarachnoid hemorrhage (SAH), multiple sclerosis (MS), Friedreich's Ataxia and Adrenoleukodystrophy.

T. The compound and/or its tautomers for use according to any one of the previous embodiments, wherein the compound and/or its tautomers is administered in form of a pharmaceutically usable solvate and/or salt thereof.

U. A unit dosage form comprising a compound of formula (I) and/or its tautomers in an amount selected from about 100 mg, about 120 mg, about 150 mg, about 180 mg, about 200 mg, about 250 mg, about 300 mg, about 375 mg, about 400 mg, about 450 mg, about 500 mg or about 750 mg and optionally one or more pharmaceutically acceptable excipients.

V. The unit dosage form according to embodiment U, comprising an amount of about 150 mg, about 250 mg, about 300 mg or about 500 mg of a compound of formula (I) and/or its tautomer.

W. The unit dosage form according to embodiment U or V, said unit dosage form being adapted to be administered orally.

X. The unit dosage form according to any one of embodiments U, V or W, said unit dosage form being in the form of a tablet or capsule or sachet.

Y. The unit dosage form according to any one of embodiments U, V W or X, wherein the compound and/or its tautomers being in the form of a pharmaceutically usable solvate and/or salt thereof.

Z. The compound and/or its tautomers for use according to any one of one embodiments A to T comprising the administration of a unit dosage form according to any one of the embodiments U to Y.

Za. A compound of formula (I) and/or its tautomers for use in a method of treating a human subject suffering from or being at increased risk of developing a disease or condition, the method comprising the administration of an oral dose of the compound of formula (I) and/or its tautomers of about 150 mg thrice a day or about 250 mg twice a day, or about 300 mg thrice a day or 500 mg twice a day.

Further preferred embodiments of the present invention are the following:

1. A dose regimen comprising the repeated administration of one or more unit dosage forms comprising the compound of formula (I) and/or its tautomers or pharmaceutically usable solvates or salts thereof to a human subject, in a dose and at a daily dosing frequency sufficient to maintain the plasma concentration of the compound of formula (I) and/or its tautomers at steady state at at least about 35 ng/mL at trough.

2. A dose regimen according to embodiment 1, wherein the plasma concentration of the compound of formula (I) and/or its tautomers is maintained in a range of about 45 ng/mL to about 2000 ng/mL at trough.

3. A dose regimen comprising the repeated administration of one or more unit dosage forms comprising the compound of formula (I) and/or its tautomers or pharmaceutically usable solvates or salts thereof to a human subject, in a dose and at a daily dosing frequency sufficient to maintain the average plasma concentration of the compound of formula (I) and/or its tautomers over a period of 24 hours at steady state at at least about 155 ng/mL.

4. A dose regimen according to embodiment 3, wherein the average plasma concentration of the compound of formula (I) and/or its tautomers is maintained over a period of 24 hours in a range of about 270 ng/mL to about 2000 ng/mL.

5. A dose regimen comprising the repeated administration of one or more unit dosage forms comprising the compound of formula (I) and/or its tautomers or pharmaceutically usable solvates or salts thereof to a human subject, in a dose and at a daily dosing frequency sufficient to maintain at steady state a plasma C max of the compound of formula (I) and/or its tautomers of about 1650 to about 7390 ng/mL.

6. A dose regimen comprising the repeated administration of one or more unit dosage forms comprising the compound of formula (I) and/or its tautomers or pharmaceutically usable solvates or salts thereof to a human subject, in a dose and at a daily dosing frequency sufficient to maintain at steady state a plasma AUC over 24 hours of the compound of formula (I) and/or its tautomers of about 13850 to about 90500 ng*h/mL.

7. A dose regimen according to one of the aforementioned embodiments, wherein the dose to be administered at said daily dosing frequency is orally administered and is in the range of about 150 mg to about 500 mg.

8. A dose regimen according to embodiment 5, wherein the plasma C max of the compound of formula (I) and/or its tautomers is about 3580 to about 7390 ng/mL ng/mL.

9. A dose regimen according to embodiment 6, wherein the plasma AUC over 24 hours of the compound of formula (I) and/or its tautomers is about 30150 to about 90500 ng*h/mL.

10. A dose regimen according to one of the aforementioned embodiments, wherein the dose to be administered at said daily dosing frequency is orally administered and is in the range of about 300 mg to about 500 mg.

11. A dose regimen according to the aforementioned embodiments, wherein the repeatedly administered dose remains constant.

12. A dose regimen according to the aforementioned embodiments, wherein the daily dosing frequency is twice a day or thrice a day.

13. A dose regimen according to the aforementioned embodiments, wherein the daily dose of the compound of formula (I) and/or its tautomers is about 240 mg to about 1500 mg per day, when orally administered.

14. A dose regimen according to the aforementioned embodiments, comprising the administration of an oral dose of the compound of formula (I) and/or its tautomers of about 150 mg thrice a day or about 250 mg twice a day, or about 300 mg thrice a day or 500 mg twice a day.

15. Method of treatment of a condition selected from neurological disorders or neurodegenerative diseases, diabetes, cancer, cardiovascular diseases and stroke, comprising the dose regimens according to embodiments 1 to 14.

16. Method of treatment of a condition selected from neurological disorders or neurodegenerative diseases, diabetes, cancer, cardiovascular diseases and stroke, comprising the administration of an oral dose of the compound of formula (I) and/or its tautomers to a human subject, of about 150 mg thrice a day or about 250 mg twice a day, or about 300 mg thrice a day or 500 mg twice a day.

17. A method of treatment of a condition according to embodiment 15 or 16, wherein the condition is selected from the group of one or more tauopathies, synucleinopathies and Alzheimer's disease (AD), amyotrophic lateral sclerosis (ALS), amyotrophic lateral sclerosis with cognitive impairment (ALSci), argyrophilic grain disease, behavioral variant frontotemporal dementia (bvFTD), non-fluent and semantic variant primary progressive aphasia (nfv & svPPA), Bluit disease, corticobasal degeneration (CBD), Dementia pugilistica, Dementia with Lewy Bodies (DLB), diffuse neurofibrillary tangles with calcification, Down's syndrome, Familial British dementia, Familial Danish dementia, frontotemporal dementia with parkinsonism linked to chromosome 17 (FTDP-17), frontotemporal lobar degeneration (FTLD), ganglioglioma, gangliocytoma, Gerstmann-Straussler-Scheinker disease, globular glial tauopathy, Guadeloupean parkinsonism, Hallevorden-Spatz disease (neurodegeneration with brain iron accumulation type 1), lead encephalopathy, lipofuscinosis, meningioangiomatosis, multiple system atrophy (MSA), myotonic dystrophy, Niemann-Pick disease (type C), Pallido-ponto-nigral degeneration, Parkinson's disease, Parkinson's disease dementia (PDD), Parkinsonism-dementia complex of Guam, Pick's disease (PiD), postencephalitic parkinsonism (PEP), Prion diseases (including Creutzfeldt-Jakob Disease (GJD), variant Creutzfeldt-Jakob Disease (vCJD), fatal Familial Insomnia, Kuru, progressive supercortical gliosis, progressive supranuclear palsy (PSP), pure autonomic failure, Richardson's syndrome, subacute sclerosing panencephalitis, Tangle-only dementia, tuberous sclerosis, Huntington's disease or mild cognitive impairment (MCI), Chronic traumatic encephalopathy, Primary progressive aphasia, Progressive nonfluent aphasia, Semantic dementia, Steele-Richardson-Olszewski syndrome, epilepsy, chronic and acute inflammation, Crohn disease, neuroinflammation, subarachnoid hemorrhage (SAH), multiple sclerosis (MS), Friedreich's Ataxia and Adrenoleukodystrophy.

18. A unit dosage form comprising an amount of a compound of formula (I) and/or its tautomers selected from about 100 mg, about 120 mg, about 150 mg, about 180 mg, about 200 mg, about 250 mg, about 300 mg, about 375 mg, about 400 mg, about 450 mg, about 500 mg or about 750 mg or pharmaceutically usable solvates, salts or tautomers thereof and optionally one or more pharmaceutically acceptable excipients.

19. The unit dosage form of embodiment 18, comprising an amount of about 150 mg, about 250 mg, about 300 mg or about 500 mg of a compound of formula (I) and/or its tautomers.

20. The unit dosage form of embodiment 18 or embodiment 19, which is adapted to be administered orally.

21. The unit dosage form of embodiments 18, 19 or 20, which is a tablet or capsule or sachet.

22. The unit dosage form of embodiments 18, 19, 20 or 21 for use in a dose regimen according to embodiments 1 to 14.

All the references cited herein are incorporated by reference in the disclosure of the invention hereby.

### EXAMPLES

The compound of Formula (I) is prepared according to WO/2016/030443.

### Example 1: e.g Human O-GlcNAcase Enzyme Inhibition Assay

5 μl of the appropriate concentration of a solution of inhibitor in Mcllvaine's Buffer (pH 6.5) in 2% DMSO (for a dose response curve calculation) is added into each well of a 384-well plate (Greiner, 781900). Then, 20 nM of His-Tagged hOGA and 10 μM of FL-GlcNAc (Fluorescein mono-beta-D-(2-deoxy-2-N-acetyl) glucopyranoside; Marker Gene Technologies Inc, M1485) were added to the 384-well plate for a final volume of 20 μl. After incubation for 60 min at room temperature, the reaction was terminated by the addition of 10 μL of stop buffer (200 mM glycine, pH 10.75). The level of fluorescence (λ_{exc} 485 nm; (λₑₘₘ 520 nm) was read on a PHERAstar machine. The amount of fluorescence measured was plotted against the concentration of inhibitor to produce a sigmoidal dose response curve to calculate an IC₅₀. All individual data was corrected by subtraction of the background (Thiamet G 3 uM=100% inhibition) whilst 0.5% DMSO was considered as the control value (no inhibition).

### Example 2: Pharmacodynamic Model

Total Protein O-GlcNAcylation Immunoassay (RL2 mAb, Meso Scale Electrochemiluminescence (ECL) Assay)

The test compound was administered orally to C57BL/6J mice. At defined time intervals after compound administration, typically a time ranging between 2 and 48 hours, preferably between 4 and 24 hours, mice were sacrificed by decapitation for blood collection and forebrain dissection.

Right brain hemispheres were placed in 2 ml Precellys tubes, snap frozen in dry ice and stored at −80° C. Left hemispheres were placed in 2 ml Eppendorf tubes, snap frozen in dry ice and stored at −80° C. until further processing. Blood samples were collected in Sarstedt tubes containing 35 IU of Heparin and kept at 4° C. After centrifugation for 10 min at 3800×g, 4° C., 50 μL of plasma from each sample was transferred to a 1.5 ml Eppendorf tube and stored at −80° C.

For the preparation of soluble brain protein for the immunoassay the hemispheres were homogenized in ice-cold Cytobuster reagent (71009—Merck Millipore) buffer with protease inhibitor cocktail. After centrifugation for 15 min at 17000×g at 4° C. the supernatants were transferred into polycarbonate tubes (1 ml). The supernatants were cleared by centrifugation for 1 h. at 100000×g, 4° C., and the protein concentrations were determined by using the BCA kit (23227—Pierce, Rockford, IL) according to the manufacturer's instructions.

### Total Protein O-GlcNAcylation Immunoassay:

Samples were randomised and 120 μg/ml (25 μl/well) of soluble brain protein was directly coated on a Multi-array 96-well high bind plate (L15XB-3 High bind—Meso Scale Discovery) overnight at 4° C. After washing (3× with PBS-T buffer), the plate was blocked with MSD blocker A solution for 1 h. at room temperature (RT) under agitation. After washing (3× with PBS-T buffer), the plate was incubated with 0.1 μg/ml of a mouse monoclonal antibody directed against O-GlcNAc moieties (RL2; MA1-072—Thermo Scientific) for 1 h. at RT under agitation. For the ECL assay, after washing (3× with PBS-T buffer), 1 μg/ml of a SULFO-TAG™ labeled anti-mouse secondary antibody (Meso Scale Discovery) was added and the plate was incubated for 1 h. at RT under agitation and protected from light. After washing (3× with PBS-T buffer), 150 μl/well of 1× Read Buffer T was added to the plates before reading on a Sector Imager 6000 (Meso Scale Discovery).

### Example 3: Preclinical Models

### A. In Vivo Treatment Efficacy on DSS Colitis Model:

Zhao M, et al. (2018) EMBO Mol. Med. 10: e8736

The dextran sodium sulfate (DSS) is a negatively charged sulfated polysaccharide of approximately 36-50 kDa which induces colitis when administered at 5% (weight/volume) in drinking water for several days (Okayasu I. et al. (1990) Gastroenterology 98:694-702).

Adult (balb/c) mice are randomized and are allowed to a period of acclimatization for one week. After this, the compound of formula (I) or vehicle is administered for 21 days by oral gavage, starting 2 weeks before the DSS treatment (Day −14) and continuing during DSS treatment and until the end of the experiment (Day +9). At Day 0, animals are getting access to a 5% (weight/volume) DSS solution in drinking water until Day +5. The DSS solution is then removed and replaced by drinking water for 4 more days until Day +9. From Day 0 until the end of the experiment (Day +12), animals are monitored daily for clinical signs of colitis including bodyweight loss, loose stools and/or diarrhoea and presence of occult or gross blood in the stools. At Day +12 or when animals reach humane endpoints, colons are dissected out, to measure their lengths and observe their content. A sample of distal colon is processed for histopathology. Another sample is homogenised and stored at −80° C. for tissue cytokine analysis. Treatment efficacy of the tested compound is evaluated according to Alenghat et al. 2013 (Alenghat T. et al. (2013) Nature 504: 153-157), by the determination of a disease score which included, i) body weight loss, ii) stool appearance iii) presence of blood in feces and iv) general appearance of the animal
weight loss (no loss=0; <5%=1; 5-10%=2; 10-20%=3; >20%=4);
stool (normal=0; soft, watery=1; very soft, semi-formed=2; liquid, sticky, or unable to defecate=3);
blood in feces (no blood=0; visible blood in rectum=1; visible blood on fur=2);
General appearance (normal=0; piloerection=1; lethargy and piloerection=2; motion less=4).

Histological injury and inflammation are scored as described in Gilbert et al (2012) (Gilbert S. et al. (2012) EMBO Mol. Med. 4: 109-124). Scoring parameters include edema (scale: 1-4), erosion/ulceration of the epithelial monolayer (scale: 1-4), crypt loss/damage (scale: 1-4), and infiltration of immune cells into the mucosa (scale: 1-4).

A decrease in the disease score resulting from less body weight loss, absence or less blood in feces, better stool consistency and better general appearance is observed after treatment with the compound of formula (I). By histology examination, treatment with the compound formula (I) results in a significant decrease of tissue inflammation.

### B. In Vivo Treatment Efficacy on Parkinson Disease (PD) Model (Line 61):

Levine P M. et al. (2019) Proc. Natl. Acad. Sci. USA. 116(5):1511-1519.

The transgenic mice (Line 61) which overexpresses wildtype human alpha-synuclein protein (hAsyn) under the regulatory control of the murine Thy-1 promoter is a widely used Parkinson disease (PD) animal model. With time, this model shows accumulation of hAsyn and aggregated deposits of hAsyn phosphorylated on serine 129 (pser129-Asyn) in cortical and subcortical regions of the brain, including the substantia nigra (Rockenstein E, et al. (2002) J. Neurosci. Res. 68(5):568-78). In addition, motor deficits such as lack of coordination, diminution of strength, and unbalance are also observed from 2 to 4 months of age (Fleming et al. 2004, J Neurosci 24 (42): 9434-40). Treatment effect of compound of formula (I) is based on the assessment of the behaviour (motoric performance), as well as the quantification of aggregated hAsyn and pser129-Asyn deposits by histology and by the biochemical determination of aggregated hAsyn present in the brain “insoluble fraction” of treated animals.

For this, transgenic Line 61 mice (4 weeks of age) and age/sex-matched non-transgenic littermates are first tested in the Irwin testing battery test, rotarod, wire suspension, beam walk and pasta gnawing test at baseline. Irwin test is performed to evaluate general health status (body weight, body temperature, existence of whiskers, constitution of the fur and eyes, righting reflex, neurological reflexes by eye blink), Wire suspension and vertical pole test are carried out to detect neuromuscular abnormalities. The rotarod test assesses motor coordination (measure of the latency to fall at certain speed in rpm/min). The beam walk test is used to evaluate motor coordination and balance (time to traverse the beam and numbers of errors/slips). The pasta gnawing test assesses orofacial motor deficits (number of bites and frequencies during biting episodes). Animals are daily treated by oral gavage with the compound of formula (I) for 24 weeks. During treatment period, animals undergo two additional rounds of behaviour tests (as described above) after 12 weeks and 24 weeks of treatment. At the end of the treatment period, all mice are euthanized under deep anesthesia and receive a transcardial perfusion with saline. Brains are removed and hemisected: left hemibrains are snap frozen on dry ice for biochemical analysis whilst the right hemibrains are post fixed in 4% PFA, embedded and frozen in cryomolds for histological evaluations. Right hemibrains are embedded in OCT medium and 10 μm cryosections are collected to quantify the level of hAsyn and pser129-Asyn deposits by immunohistochemistry in the hippocampus and in the cortex of treated animals.

For biochemical analysis of soluble and insoluble hAsyn, snap frozen hemibrains are homogenized in 10 volumes of lysis buffer [20 mM Tris-HCl, pH7.4, 50 mM NaCl, 1% Triton X-100, 0.2 mM Sodium-orthovanadate, protease inhibitor cocktail and phosphatase inhibitor cocktail] and incubated for 30 min on wet ice. After centrifugation (15,000 g, 60 min, 4° C.) supernatants are collected and referred as “Triton X-100 soluble fraction”. The Triton X-100-insoluble pellet is washed once in lysis buffer and resuspended in lysis buffer containing 2% sodium dodecyl sulfate (SDS). The resulting homogenate in 2% SDS is collected and referred as the “Triton X-100 insoluble fraction”. Levels of human alpha-synuclein present in “Triton-X-100 soluble fraction” and “Triton X-100 insoluble fractions” are quantified by electrochemilumiscence using a hAsyn immunosorbent assay kit (cat no. K151TGD) from MesoScale Discovery (MSD).

The treatment with the compound formula (I) is 1) showing by histology a reduction of the number of intraneuronal aggregated pser129-Asyn deposits; 2) demonstrating a diminution of insoluble hAsyn in the “Triton X-100 insoluble fraction” and 3) showing a functional benefit (motor improvement) in any of the behavioural tests among Irwin battery, rotarod, wire suspension, beam walk and/or pasta gnawing test.

### C. In Vivo Treatment Efficacy on the Acute Glaucoma Induced Ischemia-Reperfusion (I/R) Model:

Chen Y J et al. (2015), Invest Ophthalmol. Vis Sci. 56(3):1506-16

The efficacy of compound of formula (I) is evaluated in the acute glaucoma animal model. Eight-week-old male Sprague-Dawley (SD) rats weighing 250 to 300 g are housed in a temperature- and humidity-controlled animal room under a 12-hour light/12-hour dark cycle, with food and water provided ad libitum. Before any experiment, animals are allowed for one week of acclimatization. The SD rats are randomly allocated to control group and/or treatment groups. Compound of formula (I) is administrated by oral (gavage) route. Two protocols are considered, where the treatment is initiated 24 hours before or 1 hour after the induction of acute glaucoma-induced I/R injury.

To induce I/R injury, a general anesthesia is induced via i.p. injection of a mixture of 50 mg/kg of ketamine and 2 mg/kg of xylazine. Corneal analgesia is administered using a drop of topical 0.5% proparacaine hydrochloride ophthalmic solution, and pupillary dilatation is maintained with 0.5% tropicamide and 0.5% phenylephrine. After analgesia and dilation of the pupil, the anterior chamber of the left eye is cannulated with a 30-gauge needle connected to a saline reservoir at 150 cm above the eye, leading to a high intraocular pressure (IOP) of 110 mm Hg. The presence of retinal ischemia is examined by the fundus. The cannulation is lasting 60 minutes. After removing the infusion needle from the anterior chamber, the IOP returns to normal. Antibiotic ophthalmic gel with tobramycin is topically applied to the eye before and after the procedure. The rats are sacrificed 7 days after I/R injury to observe the long-term effect of the compound of formula (I) treatment. After collection, the thickness of the retinas is examined by histological staining (Mayer P, (1896), Mitt. zool. Stn. Neapel., 12, 303). The number of retinal ganglion cells is also quantified by immunohistochemistry. Finally, the function of the retina is also evaluated 7 days after I/R injury by electroretinography (ERG).

The treatment with the compound formula (I) is 1) mitigating the reduction of thickness of the retina measured by H&E staining; 2) showing an increase number of retinal ganglion cells stained by immunohistochemistry and 3) improving retinal function by analysing the different electrical responses obtained by electroretinography.

### D. In Vivo Treatment Efficacy on the Kainate-Induced Temporal Lobe Epilepsy (TLE) Model

Sanchez et al. (2019) Neurobiology of Disease 124: 531-543

Compound of formula (I) is evaluated in the kainate-induced seizure rat model. Sprague-Dawley rats are treated with intraperitoneal injection of kainate (10 mg/kg) to induce seizure or with saline as vehicle control. The severity of behavioral seizures following kainate injection is scored according to the Racine scale (Racine R. J. (1972). Electroencephalogr. Clin. Neurophysiol. 32 (3), 269-279), from less (score 1) to more severe (score 5): 1) mouth and face clonus and head nodding; 2) clonic jerks of one forelimb; 3) bilateral forelimb clonus; 4) forelimb clonus and rearing; 5) forelimb clonus with rearing and falling. The onset of status epilepticus (SE) is defined as the time from kainate injection to the occurrence of continuous seizure activity (Racine score 4-5) over a period of 4 h. Four weeks following the administration of kainate, animals undergo EEG surgery for electrodes implantation at the surface of the dura mater. Five weeks post-kainate administration a baseline (EEG) is recorded during 24 hours. Then, the animals receive a daily administration (oral gavage) of compound formula (I) for 3 consecutive days during which, cerebral activity is monitored by EEG recording. Animals are finally sacrificed ninety-six hours (4 days) post-baseline.

The treatment with the compound formula (I) is reducing the epileptic activity of treated animals which is determined by EEG and characterised by a reduction of the number of seizures, seizure duration, or interictal spike frequency (Sanchez et al. (2019) Neurobiology of Disease 124: 531-543).

E. In Vivo Effect of Compound of Formula (I) on the hTauP301L-Tg Mouse Model of Tauopathy

The hTauP301L-Tg model used in this study displays an age-dependent neuronal tauopathy which is characterized by a hyperphosphorylation of Tau (detected by AT8 and AT100) in the brainstem, in the midbrain and, to a lesser extend in the cortex and hippocampus. The hyperphosphorylated Tau shows conformational changes which lead to Tau aggregation, and the mice develop neurofibrillary tangles from the age of 6 months. Concomitant to the pathology, these mice progressively develop motoric deficits like clasping behaviour accompanied by a diminution of general mobility, and die prematurely at the age of 8-11 months (reMYND unpublished data, Terwel et al., 2005).

The treatment of hTauP301L-Tg mice with the compound of Formula (I) is 1) demonstrating a significant benefit in survival rate; 2) showing an improvement in motoric function (clasping behaviour, beam walk); 3) diminishing the extent of neuronal pathology and 4) reducing the level of aggregated and or hyperphosphorylated Tau in insoluble fractions of brain tissue samples.

### Example 4: Unit Dosage Form (Drug Product, DP) Preparation

### A. Description and Manufacturing of Capsule of Compound (I)

Compound of Formula (I) can be formulated as an immediate release capsule. The formulated product is a dry blend containing for example 100 mg compound (I) (free base equivalents) in a size 00 Swedish orange hard gelatine capsules. The qualitative and quantitative composition is given in Table 1.

TABLE 1
Qualitative and quantitative composition
of capsule of compound (I)

| | | Quantity |
|---|---|---|
| | | (mg/unit, 100 mg free |
| Component | Function | base equivalent) |
| Compound (I) (HCl salt) | Active ingredient | 110.00 |
| Microcrystalline cellulose | Filler | 192.71 |
| Croscarmellose sodium | Disintegrant | 6.92 |
| Colloidal silicon dioxide | Glidant | 1.73 |
| Magnesium stearate | Lubricant | 1.73 |
| Total (mg) | | 313.10 |
| Capsule size 00^{a} | Capsule shell | 1 unit |

^{a}HGC size 00 SWED OR.OP.

Compound (I) is a drug substance with suitable physical, biopharmaceutical, and chemical characteristics for development of an immediate release capsule formulation. It is suitably stable. The blend powder to be filled into the capsules contains common excipients: microcrystalline cellulose, croscarmellose sodium, colloidal silicon dioxide and magnesium stearate. The function and quality of these excipients are summarized in Table 1. All ingredients were used in concentrations typical for solid oral formulations. The selection of excipients, for the capsule powder, was based on compatibility studies and short-term stability studies. These studies showed no incompatibility with the chosen excipients.

For the manufacturing of the powder blend, excipients and active are sieved prior to use. Approximately half of the microcrystalline cellulose, compound (I) and colloidal silicon dioxide were blended. The blend was sieved and the remaining microcrystalline cellulose was added and the batch was homogenised with blending. Magnesium stearate was added to the blend and homogenised again.

For the encapsulation, the blend was filled into Size 00 gelatine capsules using encapsulation equipment. The capsules were de-dusted, weight-sorted and filled into HDPE bottles. Similar procedures as described above were followed to produce capsule with different strength of compound of formula (I)

### Method to Monitor Dissolution Rate by HPLC

### Dissolution by HPLC

A method to monitor the rate of dissolution has been established. Dissolution conditions are described below:
Type of dissolution apparatus: EP/USP basket
Medium: 0.1% CTAB in 0.01 M HCl
Volume: 900 mL
Temperature: 37° C.
Rotation speed: 75 rpm
Sinkers: NA
Sampling time points: 5, 10, 15, 30, 45, 60 minutes
Sampling information: Flow rate: 15 mL/min, In-line filter with 45 μm filter disk, with 1.5 mL
Filter: Spartan, 0.2 μm RC, 30 mm diameter

[symbol]

### Typical Chromatogranhic Conditions

Column: Waters Acquity UPLC BEH C18 130 Å, 50×2.1 mm, 1.7 μm particle size
Column temperature: 30° C.
Flow rate: 0.4 mL/min
Injection volume: 5 μL
Detection wavelength: UV 283 nm
Mobile phase: Mobile phase A: 0.1% TFA
   Mobile phase B: 0.1% TFA in acetonitrile

| | | Time (minutes) | | | | |
|---|---|---|---|---|---|---|
| | Mobile phase | 0.0 | 4.0 | 4.1 | 4.5 | 6.0 |
| Gradient elution: | A (%) | 95 | 0 | 0 | 95 | 95 |
| | B (%) | 6 | 100 | 100 | 5 | 5 |

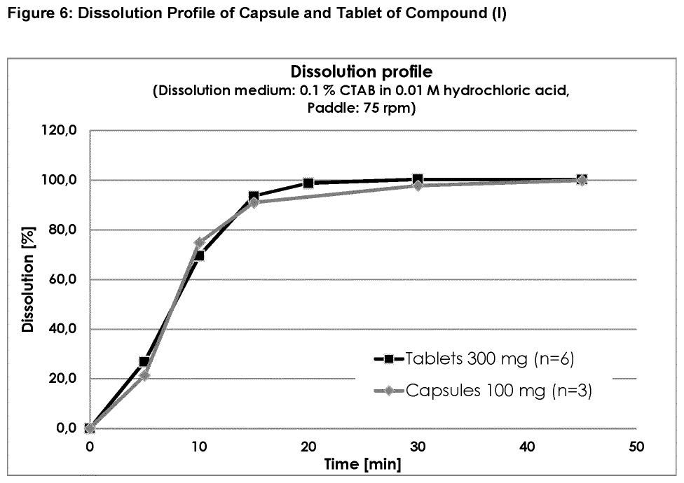

Peak retention time: Peak corresponding to compound of formula (I) elutes at about 2.1 minutes. A dissolution profile of the capsule can be found in FIG. 6.

### B. Description and Manufacturing of Film Coated Tablets of Compound (I)

Compound (I) can be formulated as film-coated tablets containing for example 300 mg compound (I) (free base equivalents) per tablet. The manufacturing unit operations leading to the formulated DP can comprise an aqueous wet granulation, followed by a tableting and aqueous film coating process. The qualitative and quantitative composition is given below in Table 2.

TABLE 2
Qualitative and quantitative composition
of film-coated tablet of compound (I),

| | | Quantity |
|---|---|---|
| Component | Function | (mg/tablet) |
| Compound (I) (HCl salt) | Active ingredient | 329.133 |
| Microcrystalline cellulose | Filler | 96.764 |
| Povidone | Binder | 58.003 |
| Purified water | Solvent | 162.922¹ |
| Croscarmellose sodium | Disintegrant | 33.000 |
| Silica, colloidal anhydrous | Glidant | 3.000 |
| Copovidone | Dry binder | 17.100 |
| Sodium stearyl fumarate | Lubricant | 13.000 |
| Tablet cores | Intermediate product | 550.00 |
| Hypromellose | Polymer | 32.500 * |
| (e.g. in Aqua Polish ®) | | |
| Macrogol | Plasticiser | Included in * |
| (e.g. in Aqua Polish ®) | | |
| Titanium dioxide | Pigment | Included in * |
| (e.g. in Aqua Polish ®) | | |
| Iron oxide | Pigment | 0.500 |
| Purified water | Solvent | 187.000¹ |
| Film-coated tablets | Bulk | 583.000 |

¹Not present in final product

Compound (I) is a drug substance with suitable physical, biopharmaceutical, and chemical characteristics for development of an immediate release oral DP formulation manufactured by conventional technologies. It is suitably stable.

The final DP contains the following excipients for the tablet core: microcrystalline cellulose, povidone, croscarmellose sodium, copovidone, silica colloidal anhydrous and sodium stearyl fumarate. A water-based film coat made from hypromellose, macrogol, titanium dioxide and iron oxide was sprayed onto the core tablets. The function and quality of every excipient used is provided in Table 2. All ingredients were used in quantities typical for oral solid formulations. The selection of excipients was further backed-up by compatibility studies and short-term stability studies of binary mixtures for key ingredients.

The typical batch formula, is presented below in Table 3 for approximately 8'000 film coated tablets for the active batch.

TABLE 3
Batch formula for film-coated tablets of compound (I)

| | | | Amount | Percentage | |
|---|---|---|---|---|---|
| Pos. | Article No. | Component | per Batch (g) | (%) | Function |
| 1 | 01 00 01 29 | Compound (I) (HCl salt) | 2650.394 | 56.46 | Active |
| | | | | | ingredient |
| 2 | 00 00 03 13 | Microcrystalline cellulose | 779.207 | 16.60 | Filler |
| | | (Avicel ® PH 102) | | | |
| 3 | 00 00 02 67 | Povidone | 467.078 | 9.95 | Binder |
| | | (Kollidion ® 25) | | | |
| 4 | 00 00 07 74 | Water for injections | 1311.954^{a} | 27.95 | Solvent |
| | | (Ampuwa ®) | | | |
| 5 | 00 00 02 09 | Croscarmellose sodium | 265.738 | 5.66 | Disintegrant |
| | | (Ac-Di-Sol ® SD-711) | | | |
| 6 | 00 00 00 38 | Silica, colloidal anhydrous | 24.158 | 0.51 | Glidant |
| | | (AEROSIL ® 200 Pharma) | | | |
| 7 | 00 00 06 66 | Copovidone | 137.700 | 2.93 | Dry binder |
| | | (Kollidon ® VA 64) | | | |
| 8 | 00 00 05 75 | Sodium stearyl fumarate | 104.684 | 2.23 | Lubricant |
| | | (Parmlub SSF) | | | |
| 9 | 10 00 00 85 | Tablet cores | 4428.959 | 94.34 | Intermediate |
| | | | | | product |
| 10 | 00 00 07 73 | Aqua Polish ® P, white 010 | 261.711 | 5.57 | Polymer |
| 11 | 00 00 07 23 | Iron oxide | 4.026 | 0.09 | Pigment |
| | | (Iron oxide Sicovit ® | | | |
| | | Red 30 E172) | | | |
| 12 | 00 00 07 74 | Water for injections | 1505.846^{a} | 32.08 | Solvent |
| | | (Ampuwa ®) | | | |
| 13 | 20 00 00 89 | Film-coated tablets | 4694.696 | 100.00 | bulk |

^{a}Not present in the final product

The different steps of the manufacturing process are described below.

### Granulation Process

Compound (I) (HCl salt), Microcrystalline cellulose and a part of the Povidone are weighed accurately as per batch formula, sieved and blended for 5-15 min in a high shear mixer granulator of suitable bowl size.
Determination of Loss on drying and bulk density (starting report values before wet massing)
Granulation solution is prepared by dissolving Povidone in purified water until a translucent solution is obtained.
The granulation solution (i.e. wet granulation) is added to the dry blend whilst continuously kneading and chopping the increasingly moist granules. Granulation time at least 3 min after all granulation solution has been added.
Sieving of the wet granules through a 3.0 mm mesh sieve
Drying of the sieved wet granules in a drying oven under the following conditions: Target: Temperature: 50-60° C., until a loss on drying of ≤3.5% is reached for the active formulation and ≤4.5% for the placebo formulation, respectively. In-Process-Control: Loss on drying: Targets: ≤3.5% (active), s 4.5% (placebo).
Sieving of the dry granules through a 1.4 mm mesh sieve
Repetition of Loss on drying determination under the same specifications plus further IPCs (bulk and tapped density, flowability).
If granules are produced in sub-batches: mixing of the fractions in a gravity blender for 10 min and repetition of the Loss on drying determination and IPCs bulk and tapped density, flowability under the same specifications after mixing.
Determine the yield.

### Blending of the Compression Mass

Adapt quantities of inter-granular phase to the yield of the granules if necessary.
Sieving of crosscarmellose sodium and silicon dioxide through a 1.4 mm mesh sieve directly into the stainless-steel drum containing the sieved dried granules.
Gravity mixing
   Target: Mixing time: 8-15 minutes
   Target: Mixer speed: 20-30 rpm
Sieving of the sodium stearyl fumarate through a 0.8 mm mesh sieve directly into the previous stainless-steel drum.
Gravity mixing
   Target: Mixing time: 3 minutes
   Target: Mixer speed: 20-30 rpm
In-Process-Controls of the compression mass:
   Loss on drying: Target: ≤4.0% (active formulation) and ≤4.5% (placebo formulation)
   Flowability Target: Free-flowing

### Tableting Process

The final compression mass is compressed into core tablets
Parameters of the tablet cores:
   Mass: Target: 550.0 mg±5% (522.5-577.5 mg)
   Length: Target: 16.6 mm+0.2 mm (16.6-16.8 mm)
   Width: Target: 7.4 mm+0.2 mm (7.4-7.6 mm)
   Height: Target: 5.2-5.8 mm
   Resistance to crushing: Target: ≥140 N
   Friability: Target: max. 1.0%
   Disintegration time: Target: ≤15 min
Dedusting of the cores and yield calculation

### Coating of Tablet of Compound (I) (HCl Salt

Whilst stirring purified water with a propeller stirrer forming a vortex, add the coating compound (e.g. Aquapolish®). Stir the suspension for 15 minutes at high speed, then reduce the speed and stir for further 45 minutes.
Put the remainder of purified water into a beaker of suitable size and homogenize (e.g. Ultra Turrax) whilst gradually adding the iron oxide and continue for around 30 min.
Add this dispersion to the continuously stirred polymer solution and continue stirring for about 30 min after which the final suspension will be passed through a 0.5 mm screen.
Transfer the tablet cores to the drum coater and warm them by inlet air (drum coater speed: interval).
   Product temperature: Target: 35-45° C.
The suspension is stirred with the propeller stirrer during the coating process and applied by means of an automatic spraying process.
   Product temperature: Target: 35-45° C.
   Atomizing air pressure: Target: 1.6-2.0 bar
   Relative spray rate: Target: max. 3.5 g/min/kg
The tablet cores are coated until the final desired weight is reached.
Parameters of the film-coated tablets:
   Appearance: pale pink, oblong without breaking notch
   Mass: Target: 583.0 mg±5% (553.9-612.2 mg)
   Length: Target: 16.6 mm+0.3 mm (16.6-16.9 mm)
   Width: Target: 7.4 mm+0.3 mm (7.4-7.7 mm)
   Height: Target: 5.4-6.0 mm (active), report value (placebo)
   Weight gain: approx. 33.0 mg/tablet core
Dry the film-coated tablets whilst operating the drum coater in interval mode at an inlet air temperature of approx. 30° C. for about 40 min.

All finished product batches are stored in tightly closed stainless steel drums, with double PE in liners with desiccants in between them.

Similar procedures as described above were followed to produce tablets with different strength of compound of formula (I)

### Method to Monitor Dissolution Rate by HPLC

A method to monitor the rate of dissolution has been established. Dissolution conditions are described below for n=6 objects to be tested with each run:
Type of dissolution apparatus: USP Paddle
Medium: 0.1% CTAB in 0.01 M HCl
Volume: 900 mL
Temperature: 37° C.
Rotation speed: 75±2 rpm
Sampling time points: 45 minutes; optional: 5, 10, 15, 30, 45 minutes
Sampling information: Flow rate: 15 mL/min, In-line filter with 45 μm filter disk, with 1.5 mL
Sample volume: 5 ml

### Typical Chromatographic Conditions

Column: Waters Acquity UPLC BEH C18 130 Å, 50×2.1 mm, 1.7 μm particle size
Column temperature: 30° C.
Flow rate: 0.3 mL/min
Injection volume: 5 μL @ approx. 0.1 mg/ml concentration
Detection wavelength: UV 283 nm
Mobile phase: 0.1% (v/v) TFA in water: 0.1% (v/v) TFA in acetonitrile=80:20 (v/v)

Peak retention time: Peak corresponding to compound (I) elutes at about 1.2 minutes.

A dissolution profile of the tablet can be found in FIG. 6.

### Example 5: Phase 1 Clinical Trials

All unit dosage forms used for the clinical trials described hereafter were prepared in accordance to example 4.

A Randomized, Double-Blind, Placebo-Controlled, Phase 1 Study to Assess the Safety, Tolerability, Pharmacokinetics and Food Effect of Single and Multiple Doses of Orally Administered Compound of Formula (I) in Healthy Adult and Elderly Subjects

The study was a single-centre, randomized, placebo-controlled single- and multiple ascending dose study in healthy male and female (no childbearing potential) volunteers aged 18-80 years. Study objectives were safety, tolerability, food effect and pharmacokinetics. Following identification of safe and tolerable single doses in healthy male subjects aged 18-55 years, separate cohorts were dosed to assess food effect. Furthermore, multiple-dose safety, tolerability, pharmacokinetics, CSF drug levels and pharmacodynamics were assessed in elderly (age 55-80 years) male or healthy non-child-bearing potential female volunteers.

The different parts of the study are illustrated schematically in the following table.

TABLE 4
Summary of the first in man study

| | |
|---|---|
| DOSE LEVEL | NUMBER OF SUBJECTS |

Part 1a: single rising doses, fasted,
healthy adult subjects

| | |
|---|---|
| 20 mg/Placebo | 6 on active/2 on placebo |
| 40 mg/Placebo | 6 on active/2 on placebo |
| 80 mg/Placebo | 6 on active/2 on placebo |
| 160 mg/Placebo | 6 on active/2 on placebo |
| 300 mg/Placebo | 6 on active/2 on placebo |
| 600 mg/Placebo | 6 on active/2 on placebo |
| 1000 mg/Placebo | 6 on active/2 on placebo |

Part 1b: single doses fasted/fed,
healthy elderly subjects

| | |
|---|---|
| 300 mg, fasted | 12 on active |
| 300 mg, fed | 12 on active |

Part 2: Healthy elderly subjects
Day 1: Twice a day (BID) dosing. Day 2:
No dosing. Day 3-11: 9 days of BID dosing.
Day 12: one dose in the morning.

| | |
|---|---|
| 100 mg BID | 6 on active/2 on placebo |
| 250 mg BID | 6 on active/2 on placebo |
| 500 mg BID | 6 on active/2 on placebo |

Part 3: single doses, fasted,
healthy adult subjects

| | |
|---|---|
| Doses from 100 mg | 6 on active; 2 doses |
| to 1000 mg | per subjects |

### Pharmacokinetics in Humans

### 1. Single Dose Pharmacokinetics

The single-dose pharmacokinetics of seven, ascending, dose levels of compound of Formula (I) (20, 40, 80, 160, 300, 600 and 1000 mg of compound (I) fasted state) were evaluated in part 1a of a randomized, placebo-controlled, time lagged, parallel/crossover group study in healthy young volunteers

Compound (I) pharmacokinetic data were measured in plasma and in urine up to 72 hours post dose. Compound (I) pharmacokinetics is characterized by consistent dose-dependent profiles which demonstrate rapid absorption, nearly dose-pro portion al increase in Cₘₐₓ from 20 to 1000 mg, and terminal half-life (t½), ranging from mean 3.9 h to mean 10.7 h at the different dose levels.

Exposure (AUC over 24 hours) showed dose proportionality at the three highest dose levels 300, 600 and 1000 mg as suggested by the dose normalized AUC not deviating more than approximately 10% at these dose levels.

In addition to the ascending dose pharmacokinetic evaluations, an evaluation of food effect was included in part 1b of the study where a separate cohort of 12 healthy elderly subjects received 300 mg of compound (I) both in fasted and fed state, which showed that food had little effect on the exposure (AUC) of compound (I), i.e. approximately 90% of the exposure in fasted state was achieved in the fed state; however absorption was delayed by approximately 3 hours and the Cₘₐₓ of compound (I) in fed state reached about 60% of the Cₘₐₓ of compound (I) in fasted state.

Comparing the pharmacokinetics of compound (I) of the healthy elderly cohort at 300 mg compound (I) in fasted state with the respective values of the healthy young cohort at 300 mg compound (I) in fasted state of part 1a, there was no relevant difference (i.e, 15% difference) of the exposure (AUC) and Cₘₐₓ.

TABLE 5
Summary (N, Arithmetic Mean (AM) and Standard Deviation (SD)) of Plasma Pharmacokinetic Parameters
of Compound (I) Following a Single Oral Dose of Compound (I) at Seven Dose Levels (20 mg,
40 mg, 80 mg, 160 mg, 300 mg, 600 mg, and 1000 mg) in Healthy Male Subjects - Part 1a

| | 20 mg | | | 40 mg | | | 80 mg | | | 160 mg | | |
|---|---|---|---|---|---|---|---|---|---|---|---|---|
| PK Parameters | N | AM | SD | N | AM | SD | N | AM | SD | N | AM | SD |
| Cₘₐₓ, ng/mL | 6 | 227 | 109 | 6 | 468 | 139 | 6 | 1370 | 912 | 6 | 1880 | 582 |
| Tₘₐₓ, h* | 6 | 0.50 (0.50, 1.50) | | 6 | 1.00 (0.50, 1.00) | | 6 | 0.50 (0.50, 1.00) | | 6 | 1.50 (0.50, 3.00) | |
| AUC_{0-inf}, | 6 | 473 | 211 | 6 | 1160 | 298 | 5^{a} | 3270 | 2050 | 5^{a} | 7010 | 1280 |
| h*ng/mL | | | | | | | | | | | | |
| t_{1/2}, h | 6 | 4.59 | 1.54 | 6 | 5.95 | 1.75 | 5^{a} | 8.84 | 5.13 | 5^{a} | 6.83 | 1.77 |

| | 300 mg | | | 600 mg | | | 1000 mg | | |
|---|---|---|---|---|---|---|---|---|---|
| PK Parameters | N | AM | SD | N | AM | SD | N | AM | SD |
| Cₘₐₓ, ng/mL | 6 | 3990 | 1550 | 6 | 7180 | 760 | 6 | 10100 | 3830 |
| Tₘₐₓ, h* | 6 | 1.25 (0.50, 3.00) | | 6 | 1.25 (0.50, 1.50) | | 6 | 1.50 (0.50, 3.00) | |
| AUC_{0-inf}, h*ng/mL | 6 | 15400 | 6480 | 6 | 33300 | 4570 | 6 | 50200 | 15800 |
| t_{1/2}, h | 6 | 7.95 | 3.57 | 6 | 6.07 | 1.48 | 6 | 6.60 | 1.85 |

*Median (min, max) reported for T_{max.}

TABLE 6
Summary (N, Arithmetic Mean and Standard Deviation) of Plasma
Pharmacokinetic Parameters of Compound (I) Following a Single
Oral Dose of 300 mg Compound (I) in Male and Female Healthy
Elderly Subjects under Fasted and Fed Conditions - Part 1b

| | Fasted Condition | | | Fed Condition | | |
|---|---|---|---|---|---|---|
| PK Parameters | N | AM | SD | N | AM | SD |
| Cₘₐₓ, ng/mL | 12 | 4160 | 1750 | 12 | 2410 | 636 |
| Tₘₐₓ, h* | 12 | 1.00 (0.50, 3.00) | | 12 | 4.00 (1.00, 6.00) | |
| AUC_{0-inf}, h*ng/mL | 12 | 17100 | 4300 | 12 | 15100 | 3030 |
| t_{1/2}, h | 12 | 7.27 | 1.72 | 12 | 7.06 | 1.76 |

*Median (min, max) reported for Tₘₐₓ.

In summary, Peak concentrations (Cₘₐₓ) and systemic exposures (AUC₀₋ₜ and AUC_{0-inf}) of plasma compound (I) clearly increased after single dose administration of compound (I) doses of 20 mg to 1000 mg.

Median Tₘₐₓ of compound (I) was between 0.50 hour and 1.50 hours, with comparable ranges of individual values for the dose levels from 20 mg to 1000 mg (ranging between 0.50 hour and 1.50 hours). Somewhat higher values were observed for the 160 mg, 300 mg, and 1000 mg dose levels (ranging between 0.50 hour and 3.00 hours).

The mean terminal half-life of compound (I) ranged from 4.59 hours to 8.84 hours over the 7 dose levels.

Dose proportionality was observed for Cₘₐₓ of compound (I) from dosing range of 20 mg to 1000 mg.

Dose proportionality was observed for AUCs of compound (I) from the dosing range of 300 mg to 1000 mg.
The Cₘₐₓ of compound (I) was approximately 42.1% lower under the fed condition compared to the fasted condition; however, overall exposure via AUC₀₋ₜ and AUC_{0-inf} of compound (I) was comparable for both fed and fasted condition when the 300 mg dose level was administered.
The high-fat, high-calorie meal administered with 300 mg compound (I) decreased Cₘₐₓ by approximately 40% and prolonged median Tₘₐₓ from 1.00 to 4.00 hours, but did not affect the extent of compound (I) absorption (AUC₀₋ₜ and AUC_{0-inf}) as compared to the fasted condition.

### 2. Multiple Dose Pharmacokinetics

The multiple-dose pharmacokinetics of three ascending, dose levels of compound (I) (100, 250, and 500 mg compound (I) given BID i.e. twice daily) were evaluated in part 2 of a randomized, placebo-controlled, time lagged, parallel group study in healthy elderly volunteers. Day 1: Twice a day (BID) dosing. Day 2: No dosing. Day 3-11: 9 days of BID dosing. Day 12: one dose in the morning.

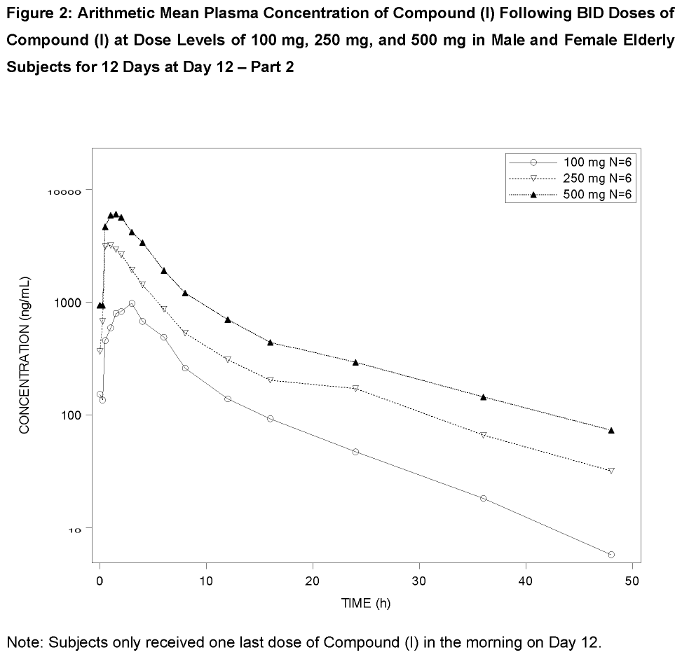

Compound (I) pharmacokinetic data were measured in plasma and in urine up to 72 hours post last dose. The compound (I) pharmacokinetics are characterized by consistent dose-dependent profiles which demonstrate rapid absorption, nearly dose-proportional increase in Cₘₐₓ and exposure (AUC) from 100 to 500 mg BID, and dose independent terminal half-life (t½), ranging from mean 3.9 h to mean 10.7 h at the different dose levels, and single dose and multiple dose conditions, respectively (FIG. 2).

Accumulation of compound (I) plasma concentrations from day 1 to day 12 was minimal and consistent with the calculated terminal half-life (t½), ie the accumulation ratios were between 1.35 and 1.38, comparing day 1 and day 12 exposure in the dosing interval.

TABLE 7
Summary (N, Arithmetic Mean and Standard Deviation)
of Plasma Pharmacokinetic Parameters of Compound (I)
Following BID Doses of 100 mg Compound (I) in Male
and Female Elderly Subjects for 12 Days - Part 2

| | Day 1 | | | Day 12 | | |
|---|---|---|---|---|---|---|
| PK Parameters | N | AM | SD | N | AM | SD |
| Cₘₐₓ, ng/mL | 6 | 1450 | 581 | 6 | 1340 | 414 |
| Tₘₐₓ, h* | 6 | 0.75 (0.50, 2.02) | | 6 | 2.25 (1.00, 6.00) | |
| Cₘᵢₙ, ng/mL | | | | 6 | 84.0 | 38.2 |
| AUC₀₋ₜₐᵤ, | 6 | 4120 | 973 | 6 | 5440 | 1340 |
| h*ng/mL | | | | | | |
| t_{1/2}, h | 3^{a, b} | 3.90 | 0.754 | 6 | 7.84 | 2.72 |
| AR*** | | | | 6 | 1.35 | 0.309 |

Note:
Cₘₐₓ and Tₘₐₓ on Day 1 is reported based on the first dose of Day 1; Cₘᵢₙ, , and AR are only reported for Da 12; Dosing interval (tau) is 12 h for multiple dose study;
*Median (min, max) reported for Tₘₐₓ;
***Accumulation ratio (AR), calculated by AUC₀₋ₜₐᵤ at Day 12/AUC₀₋ₜₐᵤ at Day 1;
^{a}t_{1/2} and λ_{z} for 2 subjects were not estimable on Day 1 due to an insufficient number of data at the terminal phase;
^{b}t_{1/2} and λ_{z} for 1 subject were excluded from the statistical analysis on Day 1 due to % AUCₑₓₜᵣₐₚ > 20% and/or the adjusted R² < 0.80.

TABLE 8
Summary (N, Arithmetic Mean and Standard Deviation)
of Plasma Pharmacokinetic Parameters of Compound (I)
Following BID Doses of 250 mg Compound (I)in Male
and Female Elderly Subjects for 12 Days - Part 2

| | Day 1 | | | Day 12 | | |
|---|---|---|---|---|---|---|
| PK Parameters | N | AM | SD | N | AM | SD |
| Cₘₐₓ, ng/mL | 6 | 3630 | 991 | 6 | 3740 | 595 |
| Tₘₐₓ, h* | 6 | 0.50 (0.50, 1.00) | | 6 | 1.00 (0.50, 2.00) | |
| Cₘᵢₙ, ng/mL | | | | 6 | 308 | 78.9 |
| AUC₀₋ₜₐᵤ, | 6 | 10700 | 2200 | 6 | 14300 | 1990 |
| h*ng/mL | | | | | | |
| t_{1/2}, h | 3^{a} | 4.74 | 0.267 | 6 | 10.2 | 2.06 |
| AR*** | | | | 6 | 1.36 | 0.246 |

Note:
Cₘₐₓ and Tₘₐₓ on Day 1 is reported based on the first dose of Day 1; Cₘᵢₙ, and AR are only reported for Day 12; Dosing interval (tau) is 12 h for multiple dose study;
*Median (min, max) reported for Tₘₐₓ;
***Accumulation ratio (AR), calculated by AUC₀₋ₜₐᵤ at Day 12/AUC₀₋ₜₐᵤ at Day 1;
^{a}t_{1/2} and λ_{z} for 3 subjects were not estimable on Day 1 due to an insufficient number of data at the terminal phase.

TABLE 9
Summary (N, Arithmetic Mean and Standard Deviation)
of Plasma Pharmacokinetic Parameters of Compound (I)
Following BID Doses of 500 mg Compound (I) in Male
and Female Elderly Subjects for 12 Days - Part 2

| | Day 1 | | | Day 12 | | |
|---|---|---|---|---|---|---|
| PK Parameters | N | AM | SD | N | AM | SD |
| Cₘₐₓ, ng/mL | 6 | 6440 | 2090 | 6 | 6670 | 1410 |
| Tₘₐₓ, h* | 6 | 1.02 (0.50, 2.00) | | 6 | 1.50 (1.00, 2.00) | |
| Cₘᵢₙ, ng/mL | | | | 6 | 743 | 299 |
| AUC₀₋ₜₐᵤ, h*ng/mL | 6 | 24300 | 5090 | 6 | 30200 | 5650 |
| t_{1/2}, h | 1^{a} | 6.19 | NA | 6 | 10.7 | 5.60 |
| AR*** | | | | 6 | 1.25 | 0.130 |

Note:
Cₘₐₓ and Tₘₐₓ on Day 1 is reported based on the first dose of Day 1; Cₘᵢₙ, and AR are only reported for Day 12; Dosing interval (tau) is 12 h for multiple dose study;
*Median (min, max) reported for Tₘₐₓ;
***Accumulation ratio (AR), calculated by AUC₀₋ₜₐᵤ at Day 12/AUC₀₋ₜₐᵤ at Day 1;
NA: Not Applicable;
^{a}t_{1/2} and λ_{z} for 5 subjects were not estimable on Day 1 due to an insufficient number of data at the terminal phase.

In summary,

### Day 1:

Cₘₐₓ, AUC₁₂₋₂₄, and AUC₀₋ₜₐᵤ of compound (I) clearly increased after BID administration of 100 mg, 250 mg, and 500 mg doses.
Dose proportionality was observed for peak exposure (Cₘₐₓ) and systemic exposures (AUC₀₋ₜₐᵤ, and AUC₁₂₋₂₄) of compound (I) at Day 1 from the dosing range of BID 100 mg to BID 500 mg doses.

### Day 12:

Cₘₐₓ, AUC₀₋₄₈, and AUC₀₋ₜₐᵤ of compound (I) clearly increased after multiple BID administration of compound (I) doses of 100 mg, 250 mg, and 500 mg.
Dose proportionality was observed for peak exposure (Cₘₐₓ) and systemic exposures (AUC₀₋ₜₐᵤ, and AUC₀₋₄₈) for compound (I) at Day 12 from the dosing range of BID 100 mg to BID 500 mg.

### 3. Cerebrospinal Fluid (CSF) Pharmacokinetics

The multiple-dose pharmacokinetics of compound (I) in CSF was assessed in a randomized, placebo-controlled, time lagged, parallel group study at three ascending dose levels of compound (I) (100, 250, and 500 mg compound (I) given BID i.e. twice daily) in healthy elderly volunteers. Day 1: Twice a day (BID) dosing. Day 2: No dosing. Day 3-11: 9 days of BID dosing. Day 12: one dose in the morning.

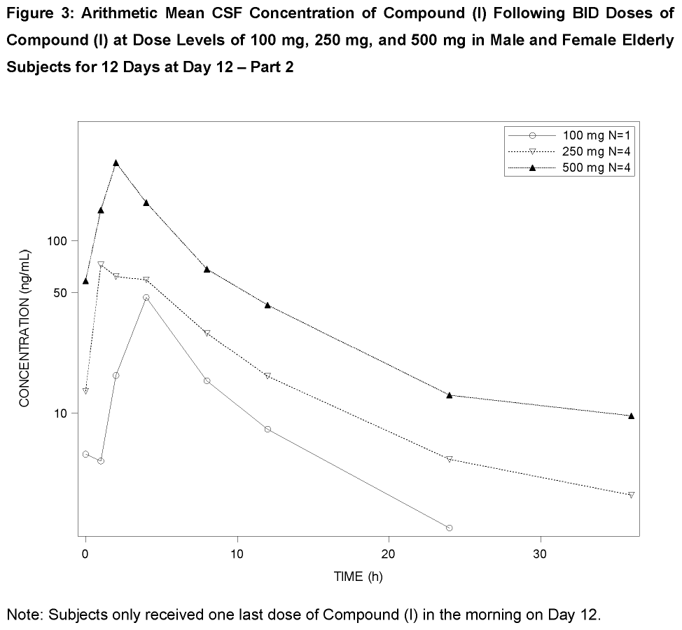

Compound (I) pharmacokinetic data were measured in CSF up to 72 hours post last dose. The compound (I) pharmacokinetics are characterized by consistent dose-dependent profiles which demonstrate rapid absorption, nearly dose-proportional increase in Cₘₐₓ and exposure (AUC) from 100 to 500 mg BID, and dose independent terminal half-life (t½), ranging from mean 3.9 h to mean 10.7 h at the different dose levels, and single dose and multiple dose conditions, respectively (FIG. 3).

TABLE 10
Summary (N, Arithmetic Mean and SD) of CSF Pharmacokinetic Parameters
of Compound (I) Following BID Doses of Compound (I) at 100 mg,
250 mg, and 500 mg in Male and Female Elderly Subjects for 12
Days with CSF Collection Starting on Day 12 - Part 2

| | 100 mg BID | | | 250 mg BID | | | 500 mg BID | | |
|---|---|---|---|---|---|---|---|---|---|
| PK Parameters | N | AM | SD | N | AM | SD | N | AM | SD |
| Cₘₐₓ, ng/mL | 1^{a} | 46.7 | NA | 4 | 92.5 | 41.3 | 4 | 282 | 11.2 |
| Tₘₐₓ, h* | 1^{a} | 4.03 (4.03, 4.03) | | 4 | 3.00 (1.00, 4.02) | | 4 | 2.02 (2.00, 2.10) | |
| AUC₀₋ₜₐᵤ, h*ng/mL | 1^{a} | 236 | NA | 4 | 485 | 138 | 4 | 1400 | 170 |
| t_{1/2}, h | 1^{a} | 5.78 | NA | 4 | 10.3 | 3.90 | 4 | 10.8 | 6.04 |

*Median (min, max) reported for Tₘₐₓ;
NA: Not Applicable;
^{a}All PK parameters for 2 subjects were excluded from the statistical analysis due to an insufficient number of data

In summary, the CSF PK parameters for compound (I) were reportable for only 1 subject at the 100 mg BID dose level due to insufficient data points. The compound (I) penetrated through the blood-brain-barrier as evidenced by their detection in cSF at Day 12 after multiple doses of compound (I). For compound (I), the CSF-to-plasma ratios for mean peak and mean systemic exposures for the 250 mg BID dose level were 2.5% and 3.4%, respectively, and for the 500 mg BID dose level, 4.2% and 4.6%, respectively.

The following summary statistics of steady-state metrics have been determined based on the above experimental results (“Mathematical Modeling of Pharmacokinetic Data”, D. W. A. Bourne, Routledge, 2018):

| Regi- | Dose | AUC (ng · h/mL) | | | | | |
|---|---|---|---|---|---|---|---|
| men | (mg) | mean | SD | CV (%) | median | min | max |
| TID | 75 | 11072 | 1908 | 17 | 10887 | 6763 | 16759 |
| | 150 | 23204 | 4345 | 19 | 22835 | 13856 | 39804 |
| | 300 | 46780 | 8237 | 18 | 45869 | 30153 | 74221 |
| BID | 125 | 12589 | 2149 | 17 | 12388 | 8381 | 19986 |
| | 250 | 25763 | 4819 | 19 | 25044 | 15995 | 40366 |
| | 500 | 52673 | 10036 | 19 | 52166 | 30750 | 90492 |

| | |
|---|---|
| Dose | Cavg (ng/mL) |

| Regimen | (mg) | mean | median | min | max |
|---|---|---|---|---|---|
| TID | 75 | 456 | 392 | 322 | 614 |
| | 150 | 951 | 827 | 647 | 1304 |
| | 300 | 1909 | 1660 | 1375 | 2657 |
| BID | 125 | 521 | 358 | 356 | 700 |
| | 250 | 1041 | 720 | 714 | 1495 |
| | 500 | 2178 | 1531 | 1488 | 3032 |

| | Dose | Cmin (ng/mL) | | | | | |
|---|---|---|---|---|---|---|---|
| Regimen | (mg) | mean | SD | CV (%) | median | min | max |
| TID | 75 | 109 | 45 | 41 | 101 | 29 | 261 |
| | 150 | 257 | 108 | 42 | 240 | 70 | 744 |
| | 300 | 533 | 210 | 39 | 499 | 185 | 1320 |
| BID | 125 | 139 | 55 | 40 | 130 | 47 | 355 |
| | 250 | 307 | 127 | 41 | 276 | 93 | 749 |
| | 500 | 654 | 270 | 41 | 617 | 176 | 1835 |

| | Dose | Cmax (ng/mL) | | | | | |
|---|---|---|---|---|---|---|---|
| Regimen | (mg) | mean | SD | CV (%) | median | min | max |
| TID | 75 | 1058 | 91 | 9 | 1053 | 862 | 1361 |
| | 150 | 2162 | 210 | 10 | 2143 | 1655 | 2925 |
| | 300 | 4348 | 386 | 9 | 4298 | 3582 | 5592 |
| BID | 125 | 1508 | 92 | 6 | 1510 | 1215 | 1791 |
| | 250 | 3027 | 195 | 6 | 3019 | 2509 | 3516 |
| | 500 | 6126 | 385 | 6 | 6133 | 5153 | 7388 |

### A Phase 1, Open-Label, Positron Emission Tomography Study in Healthy Subjects to Determine the Relationship Between Plasma Concentration and Brain Target Occupancy of Compound (I) Following a Single Oral Dose

The human brain O-linked-N-acetylglucosaminidase (O-GlcNAcase) enzyme occupancy was quantified by competitive displacement of a selective radiolabelled O-GlcNAcase inhibitor used as positron emission tomography (PET) tracer by compound of formula (I). A PET tracer can typically bear a ¹⁸F or a ¹²C as radiolabelled atom. [¹⁸F]-LSN3316612, but not limited to, may be chosen as [¹⁸F]-radiolabelled, selective O-GlcNAcase inhibitor as it has been shown to exhibit a suitable selectivity and pharmacokinetics for quantification of O-GlcNAcase enzyme in the brain in preclinical studies and as a PET tracer for human studies (Paul S. et al. (2019) J. Nucl. Med. 60: 129-134).

The primary objective of this study was to determine the brain O-GlcNAcase occupancy using [¹⁸F]-LSN3316612 Positron Emission Tomography (PET), following a single oral dose of compound (I). The secondary objective was the determination of the relationship between the plasma concentration of compound (I) and the time-course of brain O-GlcNAcase occupancy using [¹⁸F]-LSN3316612 PET, following a single oral dose of compound (I).

PET imaging data were acquired and analysed for six healthy volunteers, male. Each subject had a baseline PET scan and two post-dose PET scans. The O-GlcNAcase receptor occupancy was explored around 7 hours or 28 hours after oral doses of compound (I) (ranged from 100-1000 mg), and the corresponding estimates of occupancy ranged from 17-98%.

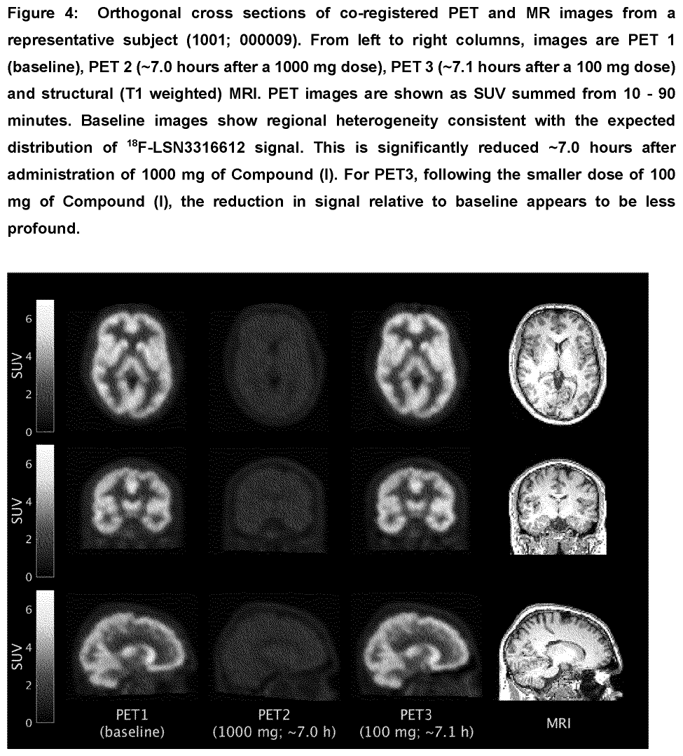

Single doses of compound (I) showed significant receptor occupancy as compared to baseline, as shown on FIG. 4.

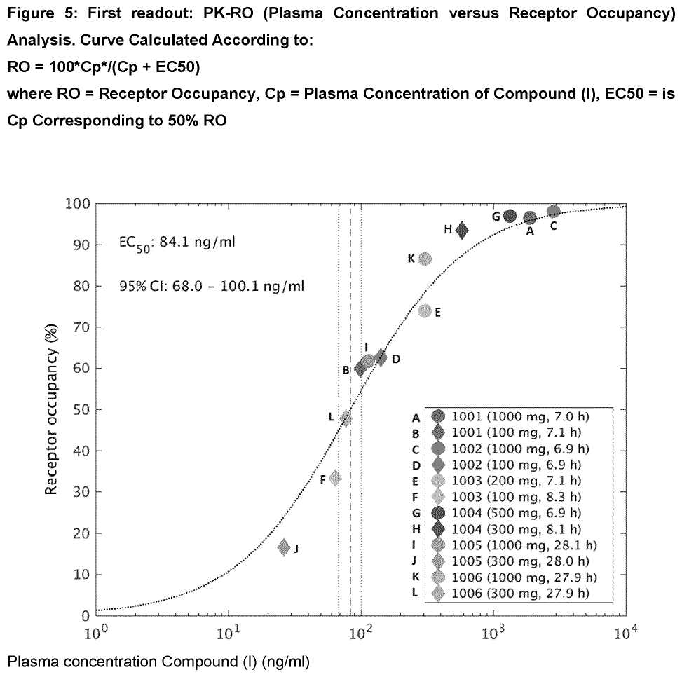

The relationship between plasma concentration of compound (I) and O-GlcNAcase was well described by a simple saturation model, with an EC₅₀ of 84.1 ng/ml (95% confidence interval: 68.0-100.1 ng/ml) (FIG. 5). The full occupancy of compound (I) was reached within the maximum dose of compound (I).

### Example 6: Phase 2 Clinical Trial

CLINICAL STUDY PROTOCOL: A Randomized, Double-blind, Placebo-Controlled Study to Evaluate the Efficacy and Safety of compound of formula (I) for the Treatment of Progressive Supranuclear Palsy

All unit dosage forms used for the clinical trials described hereafter were prepared in accordance to example 4.

PRIMARY OBJECTIVE: To evaluate the safety and tolerability of two different doses of compound (I) compared with placebo for the treatment of PSP

SECONDARY OBJECTIVES: To evaluate the following:
Pharmacokinetic (PK) parameters of compound (I)
Brain tau burden
MRI assessment of whole brain and regional midbrain atrophy
Biomarker changes in plasma and in cerebrospinal fluid (CSF)
Ability to perform daily activities
Clinical status of the subjects
Quality of life
Cognitive impairment

STUDY DESIGN: This is a randomized, multi-centre, double-blind, placebo-controlled trial to study the safety and tolerability of compound (I) in patients with probable PSP Richardson's syndrome. Approximately 40 patients (40 to 85 years of age, inclusive) are randomized in a 2:1:1 ratio to 300 mg or 150 mg or placebo TID PO with meals.

For all subjects, a screening visit is performed up to 6 weeks prior to dosing to determine eligibility. Eligible patients take investigational product (compound (I) or placebo) orally daily for about 12 weeks. Safety and tolerability data are collected at required visits to the study site. In addition, an “at home visit” for safety assessment and blood draw is organized. An open-label extension period of 52 weeks takes place upon completion of the initial double-blind treatment period. Subjects completing the double-blind treatment who demonstrate adequate compliance with the study medication are eligible to roll-over seamlessly into the open-label extension study.

TEST PRODUCT(S), DOSE AND MODE OF ADMINISTRATION: Compound (I) is administered three times daily at 300 mg or 150 mg orally (PO) or matching placebo.

PRIMARY SAFETY ENDPOINT: Frequency of treatment-emergent adverse events recorded in the three treatment arms at Week 12.

### Exploratory Efficacy Endpoints:

Change from baseline scores of the following assessments:
PSP Rating Scale (PSPRS) (28-item scale)
PSPRS subscale (10-item scale)
Cortical Basal ganglionic Functional Scale (CBFS)
PSP Functional Disability Scale (PSPFDS)
Schwab and England Activities of Daily Living Scale (SEADL)
Clinical Global Impression of Change (CGI-C)
Progressive Supranuclear Palsy Quality of Life Scale (PSP-QoL)
Montreal Cognitive Assessment (MoCA)
Dimensional Apathy Scale (DAS)
Color Trails Test Parts 1 and 2 (CTT-1 and CTT-2)
Letter Fluency Test

Change from baseline of the following parameters:
Neurodegeneration and neuroinflammation CSF biomarkers, such as neurodegeneration panel: total tau, p-tau, NfL and neuroinflammation panel
Whole brain volumes as measured by volumetric brain MRI
Regional (midbrain, frontal lobes, third ventricle) volumes as measured by volumetric brain MRI
Plasma and CSF (NfL, total tau, p-tau) concentrations

### Safety Endpoints:

Treatment-emergent adverse events (TEAEs)
Serious adverse events (SAEs)
Electrocardiograms (ECGs)
Clinical laboratory measurements
Columbia-Suicide Severity Rating Scale (C-SSRS)

### Statistical Analysis:

Analysis Population: The full analysis set includes all randomized patients. The safety set includes all patients who receive at least 1 dose of Compound (I). The per protocol set includes all patients completing the study without major protocol deviations.

The preferred dosage regimen of compound (I) are especially safe and well tolerated.

### Example 7: Phase 2 Clinical Trial, Dose Extension

The study design of this Example 7 is analogous to the study described in Example 6, a randomized, multi-centre, double-blind, placebo-controlled trial to study the safety and tolerability of compound (I) in patients with probable PSP Richardson's syndrome. Approximately 40 patients (40 to 85 years of age, inclusive) are randomized in a 2:1:1:1 ratio to 300 mg or 150 mg or 75 mg or placebo TID PO, with or without food.

The preferred dosage regimen of compound (I) are especially effective, safe and well tolerated. A dose of 75 mg TID can be used for patients that do not tolerate higher doses.

### Example 8: A Randomized, Double-Blind, Placebo-Controlled, Phase 2 Trial of Compound (I) for the Treatment of Progressive Supranuclear Palsy (PSP)

| | |
|---|---|
| Objectives | Primary Objectives: |
| | Assess the safety of Compound (I) at three dose levels for 12 weeks. |
| | Confirm the efficacy of Compound (I) at the preferred dose over 72 |
| | weeks as measured by select items from the modified Progressive |
| | Supranuclear Palsy Rating Scale (mPSPRS-10) |
| | Establish the long-term safety profile of Compound (I) |
| | Secondary Objective: |
| | Confirm the effects of Compound (I) on functional disabilities, and both |
| | clinician- and caregiver-reported global impression scales |
| | Additional Objectives: |
| | Confirm the effects of Compound (I) on pathological changes associated |
| | with PSP (whole brain and regional midbrain atrophy) |
| | Confirm the effects of Compound (I) on neurodegeneration and |
| | neuroinflammatory biomarkers in cerebrospinal fluid (CSF) and plasma |
| | Confirm of the effects of Compound (I) on cognitive function, additional |
| | health-related quality of life (HRQOL) parameters, sleep, and activities |
| | of daily living |
| | Confirm activity levels outside the clinic setting |

Design
This Phase 2 clinical trial is a global, randomized, multi-centre, double-blind, placebo-controlled
trial of Compound (I) in adults with PSP. Prospective subjects participate in the Screening
Period (up to 6 weeks); eligible subjects receive double-blind treatment (Compound (I) or
placebo) during a 72-Week Treatment Period. Following completion of the trial, eligible subjects
are invited to continue active treatment in an open-label long-term extension trial conducted
under a separate protocol.
This Phase 2 clinical trial is conducted using an adaptive trial design. An independent Data
Monitoring Committee (DMC) is responsible for trial recommendations following 3 planned
interim analyses and monitors subject safety throughout the trial.
Phase 2a
Approximately 40 eligible subjects are randomized (2:1:1:1) to receive Compound (I) at one of
3 dose levels or placebo control. Exploratory biomarker data are also analyzed by a separate
Biomarker Review Committee (BRC). During the 1^{st} interim analysis efficacy variables remain
blinded.
Phase 2b
All subjects previously randomized to active treatment during Phase 2a continue with active
treatment at the preferred dose for the remainder of the 72-Week Treatment Period; subjects
initially randomized to placebo continue on placebo for the duration of the 72-Week Treatment
Period. An additional 136 subjects are enrolled (Phase 2b) and randomized (1:1) to either
Compound (I) at the preferred dose or placebo. A second interim analysis is planned after 50%
of subjects have reached the Week 24 time point. A determination is made whether to adjust
the sample size. The BRC examine exploratory biomarker data. A third interim analysis is
planned after 50% of subjects have reached the Week 72 time point. No pauses to enrollment
are planned during the 2^{nd} and 3^{rd} interim analyses. The DMC only unblind the primary endpoint
during these interim analyses.
Number of Subjects
During Phase 2a approximately 40 subjects are randomized (2:1:1:1) to receive Compound (I)
(75 mg, 150 mg, or 300 mg) or placebo. During Phase 2b, an additional 136 subjects are
randomized (1:1) to Compound (I) at the preferred dose or placebo.
End of Trial
The end of the trial is defined as the date of the last protocol-specified visit/assessment for the
last subject participating in the trial.

| | |
|---|---|
| Interventions | Compound (I) is an immediate release film coated tablet administered as |
| | one tablet orally (PO), three times daily (TID) with or without food, at the |
| | assigned dose level (75 mg, 150 mg, or 300 mg) |
| | Placebo tablets are matched in color, size, and appearance; and are |
| | administered PO TID in the same manner as Compound (I) |

Endpoints
Safety Endpoints
Safety is assessed over 12 weeks (Phase 2a) and 72 weeks (Phase 2b) for the incidence of
treatment-emergent adverse events (TEAEs), serious adverse events (SAEs) and adverse
events of special interest (AESI), including clinically significant changes in vital signs, clinical
laboratory evaluations, physical examination findings, ECG parameters, and suicidal
ideation/behavior.
Primary Efficacy Endpoint
Change from baseline to Week 72 in the modified 10-item Progressive Supranuclear
Palsy Scale (mPSPRS-10) score
Key Secondary Endpoints
Change from baseline to Week 72 in:
Progressive Supranuclear Palsy Rating Scale (full 28-item PSPRS)
Progressive Supranuclear Palsy Clinical Deficits Scale (PSP-CDS)
Secondary Efficacy Endpoints
Change from baseline to Week 72 in:
Schwab and England Activities of Daily Living Scale (SE-ADL)
Global Impression of Change Scale (clinician (CGI-C) and caregiver (CaGI-C))
Global Impression of Severity Scale (clinician (CGI-S) and caregiver (CaGI-S))
Tertiary Endpoints
Change from baseline to Week 72 in:
Whole brain and regional (midbrain, frontal lobes, third ventricle) volumes as measured
by volumetric brain MRI
Corticobasal Degeneration Functional Scale (CBD-FS)
Progressive Supranuclear Palsy Quality of Life Scale (PSP-QoL)
Montreal Cognitive Assessment (MoCA), Version 8.2
Actigraphy
Systemic accumulation of Compound (I) (trough)
Soluble CSF and plasma biomarkers of neurodegeneration and neuroinflammation

| Statistical | Interim Analyses | |
|---|---|---|
| Considerations | 1. | Week 12 analysis (Phase 2a) of safety data and biomarkers |
| | 2. | Week 24 analysis (50% of all subjects) of safety and primary efficacy, |
| | | sample size re-estimation, biomarkers |
| | 3. | Week 72 analysis (50% of all subjects) of safety and primary efficacy |
| | | for superiority, sample size re-estimation |

According to the above trial, the preferred dosage regimen of compound (I) are especially effective, safe and well tolerated.

### REFERENCES

Boxer A L, Lang A E, Grossman M, et al. Davunetide in patients with progressive supranuclear palsy: a randomised, double-blind, placebo-controlled phase ⅔ trial. Lancet Neurol. 2014; 13(7):676-85.
Höglinger G U, Respondek G, Stamelou M, et al. (2017) Clinical diagnosis of progressive supranuclear palsy: The movement disorder society criteria. Mov Disord. a; 32:853-64.
Hoglinger G U, Schöpe J, Stamelou M, Kassubek J, Del Ser T, Boxer A L, et al. (2017) Longitudinal magnetic resonance imaging in progressive supranuclear palsy: A new combined score for clinical trials. Mov Disord. 32(6):842-52.
Stamelou M, Schöpe J, Wagenpfeil S, Del Ser T, Bang J, Lobach I Y, Luong P, et al. (2016) Power calculations and placebo effect for future clinical trials in progressive supranuclear palsy. Mov Disord. 31(5):742-7.
Tolosa E, Litvan I, Höglinger G U, et al. (2014) A phase 2 trial of the GSK-3 inhibitor tideglusib in progressive supranuclear palsy. Mov Disord. 29(4):470-8.
Wahlund L O, Barkhof F, Fazekas F, et al. (2001) A new rating scale for age-related white matter changes applicable to MRI and CT. Stroke. 32(6):1318-22.

1. A compound of formula (I) 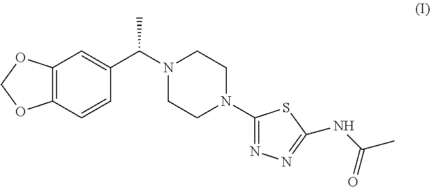 [SMILES: CC(=O)Nc1nnc(N2CCN([C@@H](C)c3ccc4c(c3)OCO4)CC2)s1]
and/or its tautomers for use in a method of treating a human subject, the method comprising the repeated administration of one or more unit dosage forms comprising the compound of formula (I) and/or its tautomers in a dose and at a daily dosing frequency sufficient to maintain the plasma concentration of the compound of formula (I) and/or its tautomers at steady state at at least about 35 ng/mL at trough.

2. The compound and/or its tautomers for use according to claim 1, wherein the plasma concentration of the compound of formula (I) and/or its tautomers is maintained in a range of about 45 ng/mL to about 2000 ng/mL at trough.

3. A compound of formula (I) 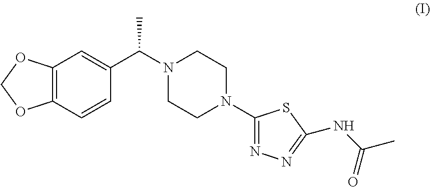 [SMILES: CC(=O)Nc1nnc(N2CCN([C@@H](C)c3ccc4c(c3)OCO4)CC2)s1]
and/or its tautomers for use in a method of treating a human subject, the method comprising the repeated administration of one or more unit dosage forms comprising the compound of formula (I) and/or its tautomers in a dose and at a daily dosing frequency sufficient to maintain the average plasma concentration of the compound of formula (I) and/or its tautomers over a period of 24 hours at steady state at at least about 155 ng/mL.

4. The compound and/or its tautomers for use according to claim 3, wherein the average plasma concentration of the compound of formula (I) and/or its tautomers is maintained over a period of 24 hours in a range of about 270 ng/mL to about 2000 ng/mL.

5. A compound of formula (I) 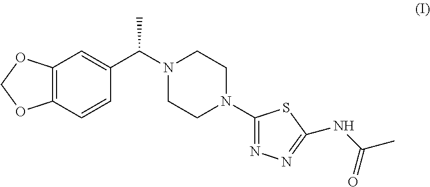 [SMILES: CC(=O)Nc1nnc(N2CCN([C@@H](C)c3ccc4c(c3)OCO4)CC2)s1]
and/or its tautomers for use in a method of treating a human subject, the method comprising the repeated administration of one or more unit dosage forms comprising the compound of formula (I) and/or its tautomers in a dose and at a daily dosing frequency sufficient to maintain at steady state a plasma C max of the compound of formula (I) and/or its tautomers of about 1650 to about 7390 ng/mL.

6. A compound of formula (I) 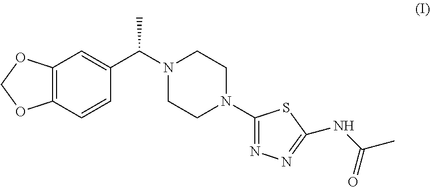 [SMILES: CC(=O)Nc1nnc(N2CCN([C@@H](C)c3ccc4c(c3)OCO4)CC2)s1]
and/or its tautomers for use in a method of treating a human subject, the method comprising the repeated administration of one or more unit dosage forms comprising the compound of formula (I) and/or its tautomers in a dose and at a daily dosing frequency sufficient to maintain at steady state a plasma AUC over 24 hours of the compound of formula (I) and/or its tautomers of about 13850 to about 90500 ng*h/mL.

7. The compound and/or its tautomers for use according to any one of the previous claims, wherein the dose of the compound of formula (I) and/or its tautomers to be administered at said daily dosing frequency is orally administered and is in the range of about 150 mg to about 500 mg.

8. The compound and/or its tautomers for use according to claim 5, wherein the plasma C max of the compound of formula (I) and/or its tautomers is about 3580 to about 7390 ng/mL.

9. The compound and/or its tautomers for use according to claim 6, wherein the plasma AUC over 24 hours of the compound of formula (I) and/or its tautomers is about 30150 to about 90500 ng*h/mL.

10. The compound and/or its tautomers for use according to any one of the previous claims, wherein the dose of the compound of formula (I) and/or its tautomers to be administered at said daily dosing frequency is orally administered and is in the range of about 300 mg to about 500 mg.

11. The compound and/or its tautomers for use according to any one of the previous claims, wherein the repeatedly administered dose of the compound of formula (I) and/or its tautomers remains constant.

12. The compound and/or its tautomers for use according to any one of the previous claims, wherein the daily dosing frequency of the compound of formula (I) and/or its tautomers is twice a day or thrice a day.

13. The compound and/or its tautomers for use according to any one of the previous claims, wherein the daily dose of the compound of formula (I) and/or its tautomers is about 240 mg to about 1500 mg per day, when orally administered.

14. The compound and/or its tautomers for use according to any one of the previous claims, comprising the administration of an oral dose of the compound of formula (I) and/or its tautomers of about 150 mg thrice a day or about 250 mg twice a day, or about 300 mg thrice a day or about 500 mg twice a day.

15. The compound and/or its tautomers for use according to any one of the previous claims, wherein the human subject suffers from a disease or condition, or is at increased risk of developing a disease or condition.

16. The compound and/or its tautomers for use according to claim 15, wherein the disease or condition is selected from one or more proteinopathies.

17. The compound and/or its tautomers for use according to claim 15 or 16, wherein the disease or condition is selected from neurological disorders or neurodegenerative diseases, sleep disorders, neuropsychiatric conditions, diabetes, cancer, cardiovascular diseases and stroke.

18. The compound and/or its tautomers for use according to any one of claims 15, 16 or 17, wherein the disease or condition is selected from the group of one or more tauopathies, synucleinopathies and Alzheimer's disease, amyotrophic lateral sclerosis, amyotrophic lateral sclerosis with cognitive impairment, argyrophilic grain disease, behavioral variant frontotemporal dementia, non-fluent and semantic variant primary progressive aphasia, Bluit disease, corticobasal degeneration, Dementia pugilistica, Dementia with Lewy Bodies, diffuse neurofibrillary tangles with calcification, Down's syndrome, Familial British dementia, Familial Danish dementia, frontotemporal dementia with parkinsonism linked to chromosome 17, frontotemporal lobar degeneration, ganglioglioma, gangliocytoma, Gerstmann-Straussler-Scheinker disease, globular glial tauopathy, Guadeloupean parkinsonism, Hallevorden-Spatz disease, lead encephalopathy, lipofuscinosis, meningioangiomatosis, multiple system atrophy, myotonic dystrophy, Niemann-Pick disease, Pallido-ponto-nigral degeneration, Parkinson's disease, Parkinson's disease dementia, Parkinsonism-dementia complex of Guam, Pick's disease, postencephalitic parkinsonism, Prion diseases, fatal Familial Insomnia, Kuru, progressive supercortical gliosis, progressive supranuclear palsy, pure autonomic failure, Richardson's syndrome, subacute sclerosing panencephalitis, Tangle-only dementia, tuberous sclerosis, Huntington's disease or mild cognitive impairment, Chronic traumatic encephalopathy, Primary progressive aphasia, Progressive nonfluent aphasia, Semantic dementia, Steele-Richardson-Olszewski syndrome, epilepsy, chronic and acute inflammation, Crohn disease, neuroinflammation, subarachnoid hemorrhage, multiple sclerosis, Friedreich's Ataxia and Adrenoleukodystrophy.

19. The compound and/or its tautomers for use according to any one of the previous claims, wherein the compound and/or its tautomers is administered in form of a pharmaceutically usable solvate and/or salt thereof.

20. The compound and/or its tautomers for use according to claim 19, wherein the compound and/or its tautomers is administered in form of its hydrochloride salt.
